SEQUENCE LISTING (69 sequences)

SEQ ID NO: 1 - DNA, 1185 bp, HSV
atggggggggctgccgccaggttgggggccgtgattttgtttgtcgtcatagtgggcctccatggggtccgcagcaaatatgccttggtggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacataccgtcgatccaggacgccgcgacgccttaccatcccccggccaccccgaacaacatgggcctgatcgccggcgcggtgggcggcagtctcctggcagccctggtcatttgcggaattgtgtactggatgcgccgccacactcaaaaagccccaaagcgcatacgcctcccccacatccgggaagacgaccagccgtcctcgcaccagcccttgttttactag

SEQ ID NO: 2 - protein, 394 aa, HSV
MGGAAARLGAVILFVVIVGLHGVRSKYALVDASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMRRHTQKAPKRIRLPHIREDDQPSSHQPLFY

SEQ ID NO: 3 - DNA, 342 bp, Mus musculus
cagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcaccctggggatacccgagcacacgtaccgctcccgcgtcaccctctccaaccagccctatatcaaggtccttaccctagccaacttcaccaccaaggatgagggcgactacttttgtgagcttcgagtctcgggcgcgaatcccatgagctccaataaaagtatcagtgtgtatagagacaagctggtcaagtgtggcggc

SEQ ID NO: 4 - protein, 114 aa, Mus musculus
QKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGG

SEQ ID NO: 5 - DNA, 315 bp, HSV
tacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacataccgtcgatccaggacgccgcgacgccttaccatcccccggccaccccgaacaacatgggcctgatcgccggcgcggtgggcggcagtctcctggcagccctggtcatttgcggaattgtgtactag

SEQ ID NO: 6 - protein, 104 aa, HSV
YSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVY

SEQ ID NO: 7 - DNA, 3589 bp, Homo sapiens
  CDS (86)..(1540)
actggcggccatggaactcaccggtaatagaggacacatctcttaactgggttgctctaagaactgatgtctaaaccgtctcagcatggcctgtagaggaggagctgggaatggccaccgtgcctcagctacactctctcgggttagccctggaagtctttacacatgtagaacccgtacccataatatatgcatggtatctgactttttctacccaaatatgggaggcgtggaaagccacatttaccagctctctcagtgcctgattgaaagagggcataaggttataattgtcacccatgcttatggaaatcgaaaaggcatccgttacctcaccagtggcctcaaagtctattacttgcctctgaaagtcatgtacaaccagtctacagccacgaccctctttcacagtctgccattgctcaggtacatatttgttcgggagagagtcacgataatccattcacatagttctttttctgctatggcccatgatgctctcttccacgccaagacaatggggcttcagacagtcttcacggaccattccctttttggatttgctgatgtcagctcggtgcttacaaacaagcttctaaccgtgtctctttgtgatacaaaccacatcatttgtgtgtcttatactagtaaggaaaatactgtactaagagcagcactgaatcctgaaatagtgtccgtcattcctaatgctgtagatcctactgacttcactccagacccatttagaaggcatgatagtataactattgttgttgtcagcagacttgtttacagaaaagggatcgatttgcttagtggtataatacctgaactctgtcagaaatatccagatttaaatttcataattggaggagagggaccaaagagaatcattttggaagaagttcgggaaagataccagctgcatgacagggtgcgtcttttgggagctttagaacacaaggatgttagaaatgtcttagttcaaggacatatttttctgaatacctcccttactgaagcattctgcatggcgatcgtggaagcagccagttgtggtttacaggttgtaagtaccagagttggtggaattcctgaggtgcttccagaaaaccttattattttatgtgagccttcagtaaaatctttgtgtgaaggattggaaaaggctattttccaactgaagtcagggacattgccagctccagaaaacatccataacatagtaaagactttctacacctggaggaatgttgcagaaagaactgaaaaggtatatgaccgggtatcagtggaagctgtgttgccaatggacaaacgactggacagacttatttctcactgcggcccagtaacaggctacatctttgctttgttggcagttttcaacttcctcttcctcattttcttgagatggatgactccagattctatcattgatgttgcaatagatgccactgggccacggggtgcctggactaataactattctcacagtaaaagagggggtgagaataatgagatatctgaaaccaggtagaaggaagcctagattgtaagattttaaacatttgtaatagttctataaagactatggaaaataaccttgcttttggggggtttttgtttttttagagttaatttagtaagttatgctacctctatatcattcaatattttctgttgaggaaagataaaaatgtatgcaattcctgagtgtagaaacttcttgcacttatttaaaatttaggagagaacatttaagccactcaggtatgcaatttttcagactactgaaatccctgtagcagagatgttttaacattatattttgagagctttgggtgctgaagggccaaacgttttctgggcattttttggccagtttttaatgtaacaccattagacactcaccagatgtttacaagttttctttaggggaactacaacaattatatgaactgttttatatcatgttcatatacatttattaggaatctaaatcatgtctttgaacatttattaggttcactcagtaggtgttacatgtaattaacaggttccttgagtaagatagtccatcagttaccagcacattttgaacccctgctctgtgtagaatgttgaactagatgcttcccgccattaaggaccaggggtgcattcactctttgtttaccattcaaatggcttacttcatcataattgtggttgatatgagatcaatatccaacatgccaaaaatgctcatgccagttaatgccaggaaaaaaatcaccgacacactactagtactttgttcctgttgtatgcattctcctaggtagagcctccatcttcagttgtgtttgtgaaggtattttttgctttttaaatactggggaccgatatcactgttgatagtgcagagaaaccctccacatttttcagtgcataattgagttttctataaatgccttcgtgttttctgagcagaatgtacgaggtgtgccatcccaaaaccagctgctaccctgtccttttaatgtaagtcactccccttcactgtggcctcgctgatgtctgataagtattgtcagtgtgcaaaaggctttacttcagaatggtttatttatagcaaactaagttgaaaattttagaaacagtctttgtgggtggatgttattaactgtcattgttgttgcccagagccatgggttttttaaccccaaattatccacatggtgtgtattatgaattctttgaactcttaaggtttttgtgagaaaaggactgtgaattcaaaacaataaggcacttgtgggtgcactacatagattctgacagtgttgtgattctgtataggatttttaaaaatgacaacattcacaaaatttattactttttaaaaaataacatgcctattaactggttgcactgatataaaagaaatatatttgtgttttgtttgtactaaaatgcaaaagcaagagtgcaatttttaaaatctagaagttaggggttttgttggagaaaaatggactgatctttaaactattcagtcttactgggatttttatgcatagaaactcacatataaacatgaaataaacagtgccagtattcataggaaagtgagaaactgtaatatttggccattattctattcaacaggttttagaggcatgccaccattttttccttatatttttgcttaatttttttaaattgtcatttaattcttaaactgtcatttatttgagatggaaataagatctaaagttagttgcctttgcctgtaaaacatgtgatttgcaaattattattttcctttttttttaacaaatggaagtaaatttgtttcacgtaaatcttaattttcaacctttctggataccttaattgtaactgtcagtttgcactggtcggtatatggaaacacattgctctaccctgctacttagttgattttaaagtgaatttacagtgatgagaaatttgtgaaaaatatattgtatttcttttgatgtttcaaaaggttgcctatgaaaaactgatttgttaaaacatgctacatgtccaaaaataaagaccagaatgacattttgat

SEQ ID NO: 8 - protein, 484 aa, Homo sapiens
MACRGGAGNGHRASATLSRVSPGSLYTCRTRTHNICMVSDFFYPNMGGVESHIYQLSQCLIERGHKVIIVTHAYGNRKGIRYLTSGLKVYYLPLKVMYNQSTATTLFHSLPLLRYIFVRERVTIIHSHSSFSAMAHDALFHAKTMGLQTVFTDHSLFGFADVSSVLTNKLLTVSLCDTNHIICVSYTSKENTVLRAALNPEIVSVIPNAVDPTDFTPDPFRRHDSITIVVVSRLVYRKGIDLLSGIIPELCQKYPDLNFIIGGEGPKRIILEEVRERYQLHDRVRLLGALEHKDVRNVLVQGHIFLNTSLTEAFCMAIVEAASCGLQVVSTRVGGIPEVLPENLIILCEPSVKSLCEGLEKAIFQLKSGTLPAPENIHNIVKTFYTWRNVAERTEKVYDRVSVEAVLPMDKRLDRLISHCGPVTGYIFALLAVFNFLFLIFLRWMTPDSIIDVAIDATGPRGAWTNNYSHSKRGGENNEISETR

SEQ ID NO: 9 - DNA, 26 bp, Artificial sequence
  Synthetic Primer
gtcctcgagtctgcagcatggcctgt

SEQ ID NO: 10 - DNA, 23 bp, Artificial sequence
  Synthetic Primer
tagggatccttctacctggtttc

SEQ ID NO: 11 - DNA, 45 bp, Artificial sequence
  Synthetic Primer
ccgcaaatcccaccaaactggtagctcgagtcgatccaggacgaa

SEQ ID NO: 12 - DNA, 24 bp, Artificial sequence
  Synthetic Primer
ataagcttgccgaaaaagctgtgg

SEQ ID NO: 13 - DNA, 31 bp, Artificial sequence
  Synthetic Primer
gtggcgccggatatccccaccaaactggcac

SEQ ID NO: 14 - DNA, 24 bp, Artificial sequence
  Synthetic Primer
ataagcttgccgaaaaagctgtgg

SEQ ID NO: 15 - DNA, 29 bp, Artificial sequence
  Synthetic Primer
aggatatctacagcttgaagatcgccggg

SEQ ID NO: 16 - DNA, 32 bp, Artificial sequence
  Synthetic Primer
tgctcgagtcaccagtttggtgggatttgcgg

SEQ ID NO: 17 - DNA, 714 bp, Homo sapiens
atggcgctgaggcggccaccgcgactccggctctgcgctcggctgcctgacttcttcctgctgctgcttttcaggggctgcctgataggggctgtaaatctcaaatccagcaatcgaaccccagtggtacaggaatttgaaagtgtggaactgtcttgcatcattacggattcgcagacaagtgaccccaggatcgagtggaagaaaattcaagatgaacaaaccacatatgtgttttttgacaacaaaattcagggagacttggcgggtcgtgcagaaatactggggaagacatccctgaagatctggaatgtgacacggagagactcagccctttatcgctgtgaggtcgttgctcgaaatgaccgcaaggaaattgatgagattgtgatcgagttaactgtgcaagtgaagccagtgacccctgtctgtagagtgccgaaggctgtaccagtaggcaagatggcaacactgcactgccaggagagtgagggccacccccggcctcactacagctggtatcgcaatgatgtaccactgcccacggattccagagccaatcccagatttcgcaattcttctttccacttaaactctgaaacaggcactttggtgttcactgctgttcacaaggacgactctgggcagtactactgcattgcttccaatgacgcaggctcagccaggtgtgaggagcaggagatggaagtctat

SEQ ID NO: 18 - protein, 238 aa, Homo sapiens
MALRRPPRLRLCARLPDFFLLLLFRGCLIGAVNLKSSNRTPVVQEFESVELSCIITDSQTSDPRIEWKKIQDEQTTYVFFDNKIQGDLAGRAEILGKTSLKIWNVTRRDSALYRCEVVARNDRKEIDEIVIELTVQVKPVTPVCRVPKAVPVGKMATLHCQESEGHPRPHYSWYRNDVPLPTDSRANPRFRNSSFHLNSETGTLVFTAVHKDDSGQYYCIASNDAGSARCEEQEMEVY

SEQ ID NO: 19 - DNA, 41 bp, Artificial sequence
  Synthetic Primer
gactgtaagcttgcccgcgtagatggcgctgaggcggccac

SEQ ID NO: 20 - DNA, 34 bp, Artificial sequence
  Synthetic Primer
cgtcaagatatcatagacttccatctcctgctcc

SEQ ID NO: 21 - DNA, 69 bp, Mus musculus
atgaacccagccatcagcgtcgctctcctgctctcagtcttgcaggtgtcccgagggcagaaggtgacc

SEQ ID NO: 22 - protein, 23 aa, Mus musculus
MNPAISVALLLSVLQVSRGQKVT

SEQ ID NO: 23 - DNA, 32 bp, Artificial sequence
  Synthetic Primer
caagtcaagcttggcaccatgaacccagccat

SEQ ID NO: 24 - DNA, 40 bp, Artificial sequence
  Synthetic Primer
atgaacggatcccctctagaggtcaccttctgaaatcggg

SEQ ID NO: 25 - DNA, 1236 bp, Rubella virus
gaggaggctttcacctacctctgcactgcaccggggtgcgccactcaagcacctgtccccgtgcgcctcgctggcgtccgttttgagtccaagattgtggacggcggctgctttgccccatgggacctcgaggccactggagcctgcatttgcgagatccccactgatgtctcgtgcgagggcttgggggcctgggtacccgcagccccttgcgcgcgcatctggaatggcacacagcgcgcgtgcaccttctgggctgtcaacgcctactcctctggcgggtacgcgcagctggcctcttacttcaaccctggcggcagctactacaagcagtaccaccctaccgcgtgcgaggttgaacctgccttcggacacagcgacgcggcctgctggggcttccccaccgacaccgtgatgagcgtgttcgcccttgctagctacgtccagcaccctcacaagaccgtccgggtcaagttccatacagagaccaggaccgtctggcaactctccgttgccggcgtgtcgtgcaacgtcaccactgaacacccgttctgcaacacgccgcacggacaactcgaggtccaggtcccgcccgaccccggggacctggttgagtacattatgaattacaccggcaatcagcagtcccggtggggcctcgggagcccgaattgccacggccccgattgggcctccccggtttgccaacgccattcccctgactgctcgcggcttgtgggggccacgccagagcgcccccggctgcgcctggtcgacgccgacgaccccctgctgcgcactgcccctggacccggcgaggtgtgggtcacgcctgtcataggctctcaggcgcgcaagtgcggactccacatacgcgctggaccgtacggccatgctaccgtcgaaatgcccgagtggatccacgcccacaccaccagcgacccctggcatccaccgggccccttggggctgaagttcaagacagttcgcccggtggccctgccacgcacgttagcgccaccccgcaatgtgcgtgtgaccgggtgctaccagtgcggtacccccgcgctggtggaaggccttgcccccgggggaggcaattgccatctcaccgtcaatggcgaggacctcggcgccgtcccccctgggaagttcgtcaccgccgccctcctcaacacccccccgccctaccaagtcagctgcgggggcgagagcgatcgcgcgagcgcgggtcat

SEQ ID NO: 26 - protein, 412 aa, Rubella virus
EEAFTYLCTAPGCATQAPVPVRLAGVRFESKIVDGGCFAPWDLEATGACICEIPTDVSCEGLGAWVPAAPCARIWNGTQRACTFWAVNAYSSGGYAQLASYFNPGGSYYKQYHPTACEVEPAFGHSDAACWGFPTDTVMSVFALASYVQHPHKTVRVKFHTETRTVWQLSVAGVSCNVTTEHPFCNTPHGQLEVQVPPDPGDLVEYIMNYTGNQQSRWGLGSPNCHGPDWASPVCQRHSPDCSRLVGATPERPRLRLVDADDPLLRTAPGPGEVWVTPVIGSQARKCGLHIRAGPYGHATVEMPEWIHAHTTSDPWHPPGPLGLKFKTVRPVALPRTLAPPRNVRVTGCYQCGTPALVEGLAPGGGNCHLTVNGEDLGAVPPGKFVTAALLNTPPPYQVSCGGESDRASAGH

SEQ ID NO: 27 - DNA, 32 bp, Artificial sequence
  Synthetic Primer
atgcagatctcgaggaggctttcacctacctc

SEQ ID NO: 28 - DNA, 30 bp, Artificial sequence
  Synthetic Primer
taccgatatcatgacccgcgctcgcgcgat

SEQ ID NO: 29 - DNA, 183 bp, HSV-1
tacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactgg

SEQ ID NO: 30 - protein, 61 aa, HSV-1
YSLKIAGWHGPKAPYTSTLLPPELSETPNATQPELAPEDPEDSALLEDPVGTVAPQIPPNW

SEQ ID NO: 31 - DNA, 37 bp, Artificial sequence
  Synthetic Primer
caagtcgcttggcaccatgaacccagccatcagcgtc

SEQ ID NO: 32 - DNA, 25 bp, Artificial sequence
  Synthetic Primer
tcgactcgagctaccagtttggtgg

SEQ ID NO: 33 - DNA, 32 bp, Artificial sequence; identical to SEQ ID NO: 23
  Synthetic Primer

SEQ ID NO: 34 - DNA, 30 bp, Artificial sequence
  Synthetic Primer
cagggatatccccgccacacttgaccagct

SEQ ID NO: 35 - DNA, 60 bp, HSV-1
tacagcttgaagatcgccgggtggcacgggcccaaggccccatacacgagcaccctgctg

SEQ ID NO: 36 - protein, 20 aa, HSV-1
YSLKIAGWHGPKAPYTSTLL

SEQ ID NO: 37 - DNA, 29 bp, Artificial sequence
  Synthetic Primer
aggatatctacagcttgaagatcgccggg

SEQ ID NO: 38 - DNA, 33 bp, Artificial sequence
  Synthetic Primer
cagtctcgagtcacagcagggtgctcgtgtatg

SEQ ID NO: 39 - DNA, 123 bp, HSV-1
cccccggagctgtccgagacccccaacgccacgcagccagaactcgccccggaagaccccgaggattcggccctcttggaggaccccgtggggacggtggcgccgcaaatcccaccaaactgg

SEQ ID NO: 40 - protein, 41 aa, HSV-1
PPELSETPNATQPELAPEDPEDSALLEDPVGTVAPQIPPNW

SEQ ID NO: 41 - DNA, 29 bp, Artificial sequence
  Synthetic Primer
tagtgatatccccccggagctgtccgaga

SEQ ID NO: 42 - DNA, 32 bp, Artificial sequence
  Synthetic Primer
tgctcgagtcagcagtttggtgggatttgcgg

SEQ ID NO: 43 - DNA, 123 bp, HSV-1
cccccggagctgtccgagaccccccaggccacgcagccagaactcgccccggaagaccccgaggattcggccctcttggaggaccccgtggggacggtggcgccgcaaatcccaccaaactgg

SEQ ID NO: 44 - protein, 41 aa, HSV-1
PPELSETPQATQPELAPEDPEDSALLEDPVGTVAPQIPPNW

SEQ ID NO: 45 - DNA, 40 bp, Artificial sequence
  Synthetic Primer
tagtgatatccccccggagctgtccgagaccccccaggcc

SEQ ID NO: 46 - DNA, 32 bp, Artificial sequence; identical to SEQ ID NO: 16
  Synthetic Primer

SEQ ID NO: 47 - DNA, 66 bp, HSV-1
cccccggagctgtccgagacccccaacgccacgcagccagaactcgccccggaagaccccgaggat

SEQ ID NO: 48 - protein, 22 aa, HSV-1
PPELSETPQATQPELAPEDPED

SEQ ID NO: 49 - DNA, 28 bp, Artificial sequence
  Synthetic Primer
tatgatatccccccggagctgtccgaga

SEQ ID NO: 50 - DNA, 35 bp, Artificial sequence
  Synthetic Primer
atactcgagtcaatcctcggggtcttccggggcga

SEQ ID NO: 51 - DNA, 27 bp, Homo sapiens
aggtctaaaaggggtggtggtggtggt

SEQ ID NO: 52 - protein, 9 aa, Homo sapiens
RSKRGGGGG

SEQ ID NO: 53 - DNA, 33 bp, Artificial sequence
  Synthetic Primer
caagtcaagcttggcaccatgaacccagccatc

SEQ ID NO: 54 - DNA, 63 bp, Artificial sequence
  Synthetic Primer
tgacgatatcgaattcaccaccaccaccaccccttttagacctcccgccacacttgaccagct

SEQ ID NO: 55 - DNA, 123 bp, HSV-2
ccgccggagctgtccgacaccacccaggccacgcaacccgaactcgttccggaagaccccgaggactcggccctcttagaggatcccgccggggcggtgtcttcgcagatccccccaaactgg

SEQ ID NO: 56 - protein, 41 aa, HSV-2
PPELSDTTQATQPELVPEDPEDSALLEDPAGAVSSQIPPNW

SEQ ID NO: 57 - DNA, 40 bp, Artificial sequence
  Synthetic Primer
cagtgatatcccgccggagctgtccgacaccacccaggcc

SEQ ID NO: 58 - DNA, 31 bp, Artificial sequence
  Synthetic Primer
cgagctcgaggaattctcaccagtttggggg

SEQ ID NO: 59 - DNA, 32 bp, Artificial sequence; identical to SEQ ID NO: 23
  Synthetic Primer

SEQ ID NO: 60 - DNA, 39 bp, Artificial sequence
  Synthetic Primer
agtcgatatcgaattctcacccgccacacttgaccagct

SEQ ID NO: 61 - DNA, 837 bp, Rubella virus
gcttctactacccccatcaccatggaggacctccagaaggccctcgaggcacaatcccgcgccctgcgcgcggaactcgccgccggcgcctcgcagtcgcgccggccgcggccgccgcgacagcgcgactccagcacctccggagatgactccggccgtgactccggagggccccgccgccgccgcggcaaccggggccgtggccagcgcagggactggtccagggccccgccccccccggaggagcggcaagaaactcgctcccagactccggccccgaagccatcgcgggcgccgccacaacagcctcaacccccgcgcatgcaaaccgggcgtgggggctctgccccgcgccccgagctggggccaccgaccaacccgttccaagcagccgtggcgcgtggcctgcgcccgcctctccacgaccctgacaccgaggcacccaccgaggcctgcgtgacctcgtggctttggagcgagggcgaaggcgcggtcttttaccgcgtcgacctgcatttcaccaacctgggcacccccccactcgacgaggacggccgctgggaccctgcgctcatgtacaacccttgcgggcccgagccgcccgctcacgtcgtccgcgcgtacaatcaacctgccggcgacgtcaggggcgtttggggtaaaggcgagcgcacctacgccgagcaggacttccgcgtcggcggcacgcgctggcaccgactgctgcgcatgccagtgcgcggcctcgacggcgacagcgccccgcttcccccccacaccaccgagcgcattgagacccgctcggcgcgccatccttggcgcatccgcttcggtgcc

SEQ ID NO: 62 - protein, 279 aa, Rubella virus
ASTTPITMEDLQKALEAQSRALRAELAAGASQSRRPRPPRQRDSSTSGDDSGRDSGGPRRRRGNRGRGQRRDWSRAPPPPEERQETRSQTPAPKPSRAPPQQPQPPRMQTGRGGSAPRPELGPPTNPFQAAVARGLRPPLHDPDTEAPTEACVTSWLWSEGEGAVFYRVDLHFTNLGTPPLDEDGRWDPALMYNPCGPEPPAHVVRAYNQPAGDVRGVWGKGERTYAEQDFRVGGTRWHRLLRMPVRGLDGDSAPLPPHTTERIETRSARHPWRIRFGA

SEQ ID NO: 63 - DNA, 34 bp, Artificial sequence
  Synthetic Primer
ctgacaggatccagcttctactacccccatcacc

SEQ ID NO: 64 - DNA, 33 bp, Artificial sequence
  Synthetic Primer
gtactggatatcctaggcaccgaagggatgcgc

SEQ ID NO: 65 - DNA, 31 bp, Artificial sequence
  Synthetic Primer
gtactggatatcggcaccgaagcggatgcgc

SEQ ID NO: 66 - DNA, 24 bp, Artificial sequence
  Synthetic Primer
tcggtaccatgggccccggtctgt

SEQ ID NO: 67 - DNA, 29 bp, Artificial sequence
  Synthetic Primer
tggaattcccgggggcgatggtggcgatg

SEQ ID NO: 68 - DNA, 123 bp, HSV-2
ccgccggagctgtccgacaccaccaacgccacgcaacccgaactcgttccggaagaccccgaggactcggccctcttagaggatcccgccggggcggtgtcttcgcagatccccccaaactgg

SEQ ID NO: 69 - protein, 41 aa, HSV-2
PPELSDTTNATQPELVPEDPEDSALLEDPAGAVSSQIPPNW

(19) United States
(12) Patent Application Publication
(10) Pub. No.: US 2003/0236396 A1
(43) Pub. Date: Dec. 25, 2003

(54) Secretory signal sequences and uses thereof

(76) Inventors: Nicholas Fasel, Epalinges (CH); Carole Beghdadi, Lausanne (CH); Chantal Desponds, Lussery-Villars (CH); Gary Cohen, Havertown, PA (US); Roselyn Eisenberg, Haddonfield, NJ (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/410,842

(22) Filed: Apr. 9, 2003

Related U.S. Application Data
(60) Provisional application No. 60/371,029, filed on Apr. 9, 2002.

(51) Int. Cl. C07H 21/04; C12N 7/00; C07K 14/035; C12N 7/01; C12N 15/86; C12P 21/02; C12N 5/06

(52) U.S. Cl. 536/23.72; 530/350; 435/69.3; 435/456; 435/235.1; 435/320.1; 435/325

(57) ABSTRACT

The present invention provides polypeptide sequences which, when fused to a heterologous polypeptide, promote secretion of the resulting chimeric protein, and uses thereof.

### FIELD OF THE INVENTION

The present invention relates to polypeptide sequences which, when fused to heterologous polypeptide sequences, promote secretion of the chimeric protein product.

### BACKGROUND OF THE INVENTION

The presence of specific signal sequences on polypeptides is essential to target cell surface membrane proteins into the endoplasmic reticulum (ER). Once translocated into the ER, the polypeptides can follow the secretory pathway and remain anchored in the membrane via peptidic transmembrane hydrophobic segment(s) (TM-anchor). For select polypeptides, cell surface targeting requires either a signal for the addition of the glycosylphosphatidylinositol anchor (GPI-anchor) in the ER or the association of the polypeptide with an accessory chaperone molecule.

Glycosylphosphotidylinositol (GPI), a complex molecule comprised of both carbohydrate moieties and lipid molecules, acts as a membrane anchor for many cell-surface proteins (reviewed in Kinoshita et al, Curr. Op. Chem. Biol. 4:632-38. 2000). The GPI anchor is ubiquitously represented in both prokaryotes and eukaryotes and possesses a structure highly conserved throughout evolution, with only minor changes seen from bacteria to mammals. A GPI anchor consists of: an acylated glucosamine residue linked to a phosphotidylinositol (PI) lipid molecule; mannose residues (3 for mammals, 4 in yeast) attached to the glucosamine-PI (GlcN-PI); and an ethanolamine linker bound to the third mannose and the C-terminal end of the anchored polypeptide. The metabolic pathway for generating a GPI anchor requires at least ten reaction steps for completion and an even greater number of enzymes. The elucidation of several enzymes involved in these steps was made possible through complementation screening of T cell lines lacking the Thy-1 molecule on the cell surface, and deficient in expressing additional GPI-linked proteins at the cell surface (Sugiyama et al., J. Biol. Chem. 266:12119-122. 1991).

Using cell surface expression of Thy-1 as a readout for the ability of the cell to complete the synthesis and attachment of GPI moieties to proteins, six complementation groups were identified (A, B, C, E, F, and H), their mutations indicating which steps and gene products are necessary for the successful expression of GPI-anchored polypeptides (Sugiyama et al., J. Biol. Chem. 266:12119-122. 1991). Complementation groups A, C, and H (cell lines BW/A, S49/A, PNH blood cells; TIMI/C; S49/H and 1tk-fibroblasts, respectively) show deficiencies in the first step of anchor biosynthesis, transfer of N-acetylglucosamine to PI, resulting in retention of the polypeptide in the ER and subsequent protein degradation. A key enzyme in this pathway is PIG-A, a catalytic component similar to many glycosyltransferases (Kinoshita. et al, Curr. Op. Chem. Biol. 4:632-38. 2000). Mutants in complementation group E fail to synthesize dolichyl phosphoryl mannose, which donates mannose residues to the building glycolipid. The class E mutant cell line BW/E secretes anchor-less Thy-1 into the culture medium. Class B mutants such as the S1 A/B hybridoma are defective in addition of the third mannose to the growing GPI-structure, and also secrete Thy-1 out of the cell rather than retaining it in the ER. Complementation group F mutants cannot add ethanolamine to the third mannose residue, but do, however, maintain the ability to attach ethanolamine to either mannose-1 or mannose-2. Class F mutants do not express Thy-1 on their cell surface. Once completed, the preformed GPI-anchor is then added to a protein in the ER via a transamidase reaction thereby aiding in expression of the protein at the cell surface.

For certain polypeptides, removal of the protein transmembrane-anchor allows the release of the membrane-bound polypeptide into the surrounding medium. In other instances, the truncated cell surface molecule is not secreted but is retained in the ER. To circumvent this retention problem, the addition of the carboxyl-terminus of the precursor of a GPI-anchored molecule onto the ectoplasmic region (the region within the cytoplasm of the endoplasmic reticulum) of a transmembranous protein is generally sufficient to direct GPI addition and anchoring of the hybrid molecule in the membrane (Caras et al., Science 238: 1280-83. 1987; Crise et al., J. Virol. 63:5 328-33. 1989; Tykocinski et al., Pro. Natl. Acad. Sci. USA 85: 355-59. 1988; Kaetzel et al., J. Biol. Chem.265: 15932-37. 1990; Bernasconi et al., J. Cell Sci. 109: 1195-201. 1996). Release into the medium of a GPI anchored molecule is mediated by cell surface treatment with a PI-specific phospholipase C or by co-expressing a GPI-specific phospholipase D which cleaves the anchor at a different site than phospholipase C (Scallon et al., Science 252: 446-48. 1991) (Bernasconi et al., J. Cell Sci. 109: 1195-201. 1996).

Anchoring of cell surface polypeptides via a GPI-linker requires specific signals. In addition to the signal peptide essential for translocation into the ER, GPI-anchored molecules are initially synthesized with a carboxy-terminal extension which is cleaved off and replaced by the glycolipidic anchor. The signal directing GPI addition requires three elements in the primary sequence of a GPI-anchored polypeptide for the addition of a GPI-anchor to a newly formed protein: the acceptor amino acid (ω) and the two immediately following residues (ω+1, ω+2); a hydrophobic C-terminal segment; and a spacer segment between those sites (reviewed in Yeh et al, Sem. Immunol. 6: 73-80. 1994).

The acceptor site (ω), or C-terminal cleavage site, for GPI addition requires that the amino acid residues in this site and the two immediately following residues (ω+1, ω+2) be generally small residues such as glycine, aspartic acid, alanine, asparagine, serine, or cysteine (Moran et al, J. Biol. Chem. 266: 1250-57. 1991; Gerber et al, J. Biol. Chem. 267: 12168-173. 1989). This acceptor site is followed by a 5-10 amino acid spacer sequence of hydrophilic amino acids (Beghdadi-Rais et al., J. Cell Sci. 105: 831-40. 1993), which then leads to the C-terminal hydrophobic domain of approximately 15-20 amino acid residues. However, simply cleaving the transmembrane-domain and adding an appropriate hydrophobic sequence to a polypeptide is not sufficient to induce GPI-anchoring (Caras et al, J. Cell Biol. 108: 1387-96. 1989). When the GPI anchor is not added onto the polypeptide due to either i) a deficiency in the biosynthetic pathway required for GPI anchor synthesis and attachment, or ii) a mutation in the amino acid C-terminal signal sequence on the molecule, the hydrophobic sequence is not cleaved and the molecule is rapidly degraded in the ER.

Using a truncated form of HSV-1 glycoprotein D (gD-1), a natural transmembrane protein involved in viral attachment and fusion to host cells, it was demonstrated that a specific C-terminal region of the gD polypeptide can act as a signal to direct GPI-anchor addition (Beghdadi-Rais et al., J. Cell Sci. 105:831-40. 1993). Fusion of this C-terminal domain (gDL) to the ectodomain of the Thy-1 antigen induces GPI-anchoring of the fusion polypeptide Thy-1/gDL (Beghdadi-Rais et al., J. Cell Sci. 105:831-40. 1993) to the cell membrane. To assess biochemically the mode of anchoring, GPI-deficient cell lines can serve as a simple biological assay to distinguish between GPI and TM anchoring of cell surface polypeptides. In GPI-deficient cells, TM anchored polypeptides are correctly transported to the cell surface, whereas polypeptide precursors of GPI-anchored molecules are blocked in the ER (complementation class A, C, D, F and H) or released in the medium (complementation class B). Similarly, if the signal peptide essential for GPI anchoring is not recognized by the cellular GPI-anchor addition machinery, the protein will not be transported to the cell surface but will be degraded in the ER. Thus, this expression system based on GPI-deficient cells can be used firstly to determine if the GPI-anchor process is efficient for such artificially modified molecules. Secondly, this system allows to distinguish molecules which are not degraded in the ER but reach the surface where they are anchored either via a peptidic transmembrane region or with a GPI anchor. Lastly it allows verification that some chimeric polypeptides are secreted in the medium.

The ability to alter protein expression and construct a protein with either membrane bound or secreted properties could have great effects on the generation of therapeutic compounds for prevention and/or treatment of a growing number of diseases whose cause is aberrant expression of a particular protein. Thus, there exists a need in the art to delineate polypeptide sequences involved in the regulation of protein cell surface expression and secretion, sequences that may be fused to a polypeptide of interest thereby modulating the activity of specific proteins without use of toxic materials or non-specific therapeutics. Identification of such polypeptide sequences will also provide improved methods for protein production and isolation of the chimeric from media of cell growth.

### SUMMARY OF THE INVENTION

The present invention provides purified and isolated polynucleotides encoding secretory signal polypeptides, wherein said polypeptides are non-cell surface anchoring amino terminal fragments of sequence HSV-1 gDL (SEQ ID NO: 6) lacking at least 40 carboxy terminal amino acid residues, and substitution variants thereof that retain secretory activity. In one aspect, polynucleotides of the invention consists essentially of sequence HSV-1 gDS (SEQ ID NO: 29), sequence HSV-1 gDS2-N (SEQ ID NO: 43), sequence HSV-1 gDS1 (SEQ ID NO: 35), sequence HSV-1 gDS2 (SEQ ID NO: 39), sequence HSV-1 gDS-PD (SEQ ID NO: 47), sequence HSV-2 gDS (SEQ ID NO: 68) and sequence HSV-2 gD2S2-N (SEQ ID NO: 55), as well as substitution variants of any of these sequences.

In another aspect, the invention provides polynucleotides encoding secretory polypeptides selected from the group consisting of a polynucleotide as described herein and polynucleotides consisting essentially of a polypeptide-coding region that specifically hybridizes to the secretory polypeptide-coding region of a polynucleotide of the invention under conditions that include a final wash in 0.1×SSC and 0.1% SDS at 65° C. In one aspect, the polynucleotide is selected from the group consisting of gDS2-N (SEQ ID NO: 43), gD2S2-N (SEQ ID NO: 55), and HSV2 gDS (SEQ ID NO: 68).

The invention further provides chimeric polynucleotides comprising a secretory signal encoding polynucleotide of the invention and a heterologous polypeptide coding region, wherein the chimeric polynucleotide encodes a polypeptide that is secreted and includes a secretory signal peptide. In one aspect, the chimeric polynucleotide further comprises one or more operatively-linked expression regulatory elements 5′ to the chimeric polynucleotide and a stop codon 3′ to the chimeric polynucleotide. In another aspect, the chimeric polynucleotide further comprises a polynucleotide encoding a peptide cleavage site which is positioned in-frame between the heterologous polypeptide coding region. In one aspect, the heterologous polypeptide coding region is positioned 5′ to secretory signal polypeptide coding region, and in another aspect, the secretory signal coding region is position 5′ to the heterologous polypeptide coding region.

The invention further provides expression vectors comprising a chimeric polynucleotide of the invention, as well as host cells transformed or transfected with an expression vector of the invention or a chimeric polynucleotide of the invention.

The invention also provides methods for expression of a secreted polypeptide comprising the steps of growing a host cell of the invention under conditions that permit expression and secretion of the heterologous polypeptide. In one aspect, methods of the invention further comprise the step of cleaving the secretory polypeptide from the heterologous polypeptide at the peptide cleavage site.

### DETAILED DESCRIPTION OF THE INVENTION

The present invention relates to polynucleotides encoding secretory signal polypeptide sequences which, when combined with a polynucleotide encoding a polypeptide, the resulting encoded polypeptide is secreted from a cell in which it is expressed. Polynucleotides of the invention include DNA (genomic, complementary, amplified, or synthetic) and RNA, as well as polynucleotide mimetic that, while chemically distinct from naturally occurring polynucleotides, encode a secretory signal polypeptide that can be expressed in a manner similar to a signal polypeptide encoded by a polynucleotide of the invention. In one aspect, the secretory signal polypeptide of the invention is located within glycoprotein D of herpes simplex virus 1 (HSV-1). Polynucleotides of the invention include, but are not limited to, a purified and isolated polynucleotide encoding a secretory signal polypeptide, wherein said polypeptide is a non-cell surface anchoring amino terminal fragment of a gD polypeptide designated gDL (SEQ ID NO: 6), said fragment encoded by the polynucleotide set out in: SEQ. ID NO.: 5 and lacking polynucleotide sequences encoding for at least 40 carboxy terminal amino acid residues in the polypeptide of SEQ ID NO: 6. In various aspects, the invention provides polynucleotides as set out in SEQ. ID NOs.: 29, 35, 39, 43, 47, 55, or 68; or variants thereof that encode a secretory polypeptide that retains secretory activity. The polynucleotides of the present invention also include, but are not limited to, a polynucleotide consisting essentially of a polypeptide-coding region that specifically hybridizes under stringent conditions to (a) the complement of any of the polynucleotides selected from the group consisting of SEQ. ID NO.: 5 lacking codons that encode at least 40 C-terminal amino acid residues, SEQ. ID NOs.: 29, 35, 39, 43, 47, 55, and 68; (b) a polynucleotide encoding a polypeptide selected from the group consisting of SEQ ID NO: 6 lacking at least 40 C-terminal amino acid residues, SEQ ID NOs: 30, 36, 40, 44, 48, 56 and 69; (c) polynucleotides encoding polypeptides which are “substantially equivalent” to a secretory signal polypeptide encoded by a polynucleotide of the invention; and (d) polynucleotides encoding variant polypeptides which possess the ability to signal secretion of a polypeptide to which it is attached; and (e) a polynucleotide which encodes a homolog (viral or otherwise) of any of the polypeptides recited above, wherein the polypeptide possesses the ability to signal secretion of a polypeptide to which it is attached.

The term “stringent” as used herein includes highly stringent conditions including a final wash in 0.1×SSC/0.1% SDS at 65° C.), and moderately stringent conditions (i.e., final wash in 0.2×SSC/0.1% SDS at 42° C.). In instances of hybridization of oligonucleotides that encode a secretory signal or a probe that can be used to specifically identify a polynucleotide encoding such a signal sequence, exemplary stringent hybridization conditions include washing in 6×SSC/0.05% sodium pyrophosphate at 37° C. (for 14-base oligonucleotides), 48° C. (for 17-base oligonucleotides), 55° C. (for 20-base oligonucleotides), and 60° C. (for 23-base oligonucleotides). Included within the scope of the nucleic acid sequences of the invention are nucleic acid sequence fragments of a polynucleotide of the invention that specifically hybridize under stringent conditions to any of the nucleotide sequences of the invention, or complements thereof, wherein the fragment is greater than about 5 nucleotides, preferably 7 nucleotides, more preferably greater than 9 nucleotides and most preferably greater than 17 nucleotides. Fragments of, e.g. about 15, about 17, or about 20 nucleotides or more that are selective for (i.e. specifically hybridize to any one of the polynucleotides of the invention) are contemplated.

The term “secreted” describes a protein that is transported across or through a membrane, to the exterior of the cell in which it is expressed. “Secreted” proteins include without limitation proteins which are wholly secreted (e.g., soluble proteins) from the cell in which they are expressed. “Secreted” proteins also include without limitation proteins that are transported across the membrane of the endoplasmic reticulum. “Secreted” proteins are also intended to include proteins containing non-typical signal sequences (e.g. interleukin-1 beta, see Krasney, P. A. and Young, P. R. (1992) Cytokine 4(2):134 -143) and factors released from damaged cells (e.g. interleukin-1 receptor antagonist, see Arend, W. P. et. al. (1998) Annu. Rev. Immunol. 16:27-55).

Polynucleotides according to the invention which are “substantially equivalent” include those that have, e.g., at least about 65%, at least about 70%, at least about 75%, at least about 80%, 81%, 82%, 83%, 84%, 85%, 86%, 87%, 88%, or 89%, more typically at least about 90%, 91%, 92%, 93%, or 94% and even more typically at least about 95%, 96%, 97%, 98% or 99% sequence identity to a polynucleotide described herein that maintain biological activity. As used herein, “substantially equivalent” can refer both to nucleotide and amino acid sequences, for example a mutant sequence, that varies from a reference sequence by one or more substitutions, deletions, or additions, the net effect of which does not result in an adverse functional dissimilarity between the reference and subject sequences.

“Variant” polynucleotides of the invention include naturally-occurring polynucleotides as well as chemically altered polynucleotides. Naturally-occurring polynucleotide variants of the invention are those that (i) are found in nature, e.g., in related viral and non-viral species, (ii) are related to a polynucleotide of the invention through chemical similarity as described herein and (iii) encode a polypeptide that possesses the ability to signal secretion of a polypeptide to which the encoded polypeptide is linked. Exemplary variant polynucleotides include the polynucleotide designated HSV-2 gDS (SEQ ID NO: 68), HSV-2 gD2S2-N (SEQ ID NO: 55) and HSV- 1 gDS2-N (SEQ ID NO: 43). Variants of this type and others are identified using the hybridization and probe techniques as described above.

Chemically altered, or synthetic, polynucleotide sequence variants are those that are not found in nature, and variants of this type may be prepared by methods known in the art by introducing appropriate nucleotide changes into a naturally-occurring polynucleotide to effect changes in the encoded polypeptide sequence. There are at least two variables to be considered in construction of amino acid sequence variants, including the location of the mutation and the nature of the mutation. These nucleic acid alterations can be made at sites that differ in the nucleic acids from different species (variable positions) or in highly conserved regions (constant regions). Sites at such locations will typically be modified in series, e.g., by substituting first with conservative choices (e.g., hydrophobic amino acid to a different hydrophobic amino acid) and then with more distant choices (e.g., hydrophobic amino acid to a charged amino acid), and then deletions or insertions may be made at the target site.

The term “variant” (or “analog”) therefore refers to any polypeptide differing from naturally occurring polypeptides by amino acid insertions, deletions, and substitutions, created using, e g., recombinant DNA techniques. Guidance in determining which amino acid residues may be replaced, added or deleted without abolishing activities of interest, may be found by comparing the sequence of the particular polypeptide with that of homologous peptides and minimizing the number of amino acid sequence changes made in regions of high homology (conserved regions) or by replacing amino acids with consensus sequence.

Alternatively, recombinant variants encoding these same or similar polypeptides may be synthesized or selected by making use of the “redundancy” in the genetic code as described above. As used herein, amino acid “substitutions” are the result of replacing one amino acid with another amino acid having similar structural and/or chemical properties, i.e., conservative amino acid replacements. “Conservative” amino acid substitutions may be made on the basis of similarity in polarity, charge, solubility, hydrophobicity, hydrophilicity, and/or the amphipathic nature of the residues involved. For example, nonpolar (hydrophobic) amino acids include alanine (Ala, A), leucine (Leu, L), isoleucine (Iso, I), valine (Val, V), proline (Pro, P), phenylalanine (Phe, F), tryptophan (Trp, W), and methionine (Met, M); polar neutral amino acids include glycine (Gly, G), serine (Ser, S), threonine (Thr, T), cysteine (Cys, C), tyrosine (Tyr, Y), asparagine (Asn, N), and glutamine (Gln, Q); positively charged (basic) amino acids include arginine (Arg, R), lysine (Lys, K), and histidine (His, H); and negatively charged (acidic) amino acids include aspartic acid (Asp, D) and glutamic acid (Glu, E). “Insertions” or “deletions” are preferably in the range of about 1 to 20 amino acids, more preferably 1 to 10 amino acids. The variation allowed may be experimentally determined by systematically making insertions, deletions, or substitutions of amino acids in a polypeptide molecule using recombinant DNA techniques and assaying the resulting recombinant variants for activity.

Due to the inherent degeneracy of the genetic code, other DNA sequences which encode the same, substantially the same or a functionally equivalent amino acid sequence, are embraced by the invention. These “degenerate variants” differ from a nucleic acid fragment of the present invention by nucleotide sequence but, due to the degeneracy of the genetic code, encode an identical polypeptide sequence. Various codon substitutions, such as silent changes which produce various restriction sites, may be introduced to optimize cloning into a plasmid or viral vector or expression in a particular prokaryotic or eukaryotic system. Such DNA sequences include those which are capable of hybridizing to nucleic acid sequence of the invention under stringent conditions.

In a preferred method, polynucleotides encoding the secretory signal sequences are changed via site-directed mutagenesis. This method uses oligonucleotide sequences to alter a polynucleotide to encode the desired amino acid variant, as well as sufficient adjacent nucleotides on both sides of the changed amino acid to form a stable duplex on either side of the site being changed. In general, the techniques of site-directed mutagenesis are well known to those of skill in the art and this technique is exemplified by publications such as, Edelman et al., DNA 2:183 (1983). A versatile and efficient method for producing site-specific changes in a polynucleotide sequence was published by Zoller and Smith, Nucleic Acids Res. 10:6487-6500 (1982). PCR may also be used to create amino acid sequence variants of the novel nucleic acids. When small amounts of template DNA are used as starting material, primer(s) that differs slightly in sequence from the corresponding region in the template DNA can generate the desired amino acid variant. PCR amplification results in a population of product DNA fragments that differ from the polynucleotide template encoding the polypeptide at the position specified by the primer. The product DNA fragments replace the corresponding region in the plasmid and this gives a polynucleotide encoding the desired amino acid variant. A further technique for generating amino acid variants is the cassette mutagenesis technique described in Wells et al., Gene 34:315 (1985); and other mutagenesis techniques well known in the art, such as, for example, the techniques in Sambrook et al., (1989) Molecular Cloning: A Laboratory Manual, Cold Spring Harbor Laboratory, NY), and Ausubel et al., Current Protocols in Molecular Biology.

The polynucleotides of the invention additionally include the complement of any of the polynucleotides recited above. Complementary sequences of this type are particularly useful in the identification of related sequences as described herein, as well as serving as template polynucleotides from which synthetic variants of the invention can be prepared, using, for example, polymerase chain reaction (PCR) under optimized standard conditions.

The invention further provides “chimeric polynucleotides” encoding proteins comprising a secretory signal of the invention and a heterologous amino acid sequence. As used herein, a “heterologous” polynucleotide comprises a polypeptide-coding region linked in proper reading fame (“in-frame”) via techniques described herein or otherwise known in the art, to another second protein coding sequence, wherein the first, heterologous polypeptide coding region is not naturally associated with the second polypeptide coding sequence. Specifically contemplated are chimeric polynucleotides (and “chimeric polypeptides” encoded by the polynucleotides) which are comprised of a first, heterologous polynucleotide described previously which encodes a polypeptide operably linked to a second polynucleotide of the invention. Within the chimeric polynucleotides, the term “operatively-linked” is intended to indicate that the heterologous polynucleotide and the secretory signal polynucleotide are attached in-frame with one another so that the expressed polypeptide includes both encoded sequences. The heterologous polynucleotide can be linked to the N-terminus or C-terminus of the secretory signal polynucleotide. The resulting “chimeric polypeptide” encoded by a chimeric polynucleotide of the invention is characterized by its ability to be secreted from a host cell (as described herein) without an intermediate step of being anchored to the host cell membrane. In one aspect, the polypeptide encoded by the heterologous polynucleotide sequence is not normally a secreted protein (i.e., the heterologous polypeptide is found in the cytoplasm or attached to the cell membrane of a cell in which it is expressed) without the addition of the secretory signal polypeptide. Alternatively, the polypeptide encoded by the heterologous polynucleotide is secreted from a host cell as a relatively low level compared to the level of secretion observed when the encoded heterologous polypeptide includes an additional secretory signal polypeptide sequence. In still another alternative, a heterologous polynucleotide encodes a polypeptide that has been altered in such a way that it is no longer capable of secretion from a cell in which it is expressed, even though the “unaltered” protein is secreted from the same cell type.

A chimeric polynucleotide of the invention can be produced by standard polynucleotide modification techniques. For example, DNA fragments coding different polypeptide sequences are ligated in-frame in accordance with conventional techniques, e.g., by employing blunt-ended or overhanging termini for ligation, restriction enzyme digestion to provide for appropriate termini, filling-in of cohesive ends as appropriate, alkaline phosphatase treatment to avoid undesirable joining, and enzymatic ligation. In another embodiment, the chimeric polynucleotide can be synthesized by conventional techniques including automated DNA synthesizers. Alternatively, PCR amplification of polynucleotide fragments can be carried out using anchor primers that give rise to complementary overhangs between two consecutive polynucleotide fragments that can subsequently be annealed and reamplified to generate a chimeric polynucleotide sequence (see, e.g., Ausubel, et al. (eds.) Current Protocols in Molecular Biology, John Wiley & Sons, 1992).

Chimeric polynucleotide sequences comprising the heterologous polypeptide and a secretory signal coding sequence, or functional equivalents thereof, may be used to generate recombinant DNA molecules that direct the expression of that nucleic acid, or a functional equivalent thereof, in appropriate host cells. A heterologous polynucleotide according to the invention can be joined to any of a variety of other nucleotide sequences by well-established recombinant DNA techniques (see Sambrook J et al. supra).

The invention further provides chimeric polynucleotides inserted into an expression vector for the purpose of translating the polynucleotide into polypeptide. The term “recombinant expression vehicle or vector” refers to a plasmid or phage or virus or vector, for expressing a polypeptide from a polynucleotide sequence. Expression vectors comprise a transcriptional unit comprising an assembly of (1) one or more genetic elements having a regulatory role in gene expression, for example, promoters or enhancers, (2) a structural or coding sequence which is transcribed into mRNA and translated into protein, and (3) appropriate transcription initiation and termination sequences. Structural units intended for use in yeast or eukaryotic expression systems may optionally include a leader sequence to further enhance extracellular secretion of translated protein by a host cell. Alternatively, where recombinant protein is expressed without a leader or transport sequence, it may include an amino terminal methionine residue. This residue may or may not be subsequently cleaved from the expressed recombinant protein to provide a final product. Useful include vectors, e.g., plasmids, cosmids, lambda phage derivatives, phagemids, and the like, that are well known in the art. In general, the vector contains an origin of replication functional in at least one organism, convenient restriction endonuclease sites, and a selectable marker for the host cell. Vectors according to the invention include expression vectors, replication vectors, probe generation vectors, and sequencing vectors.

In the case of a vector comprising a heterologous polypeptide of the present invention, the vector may further comprise regulatory sequences, including for example, a promoter, operably linked to the heterologous nucleotide sequence. Large numbers of suitable vectors (many of which include endogenous regulatory DNA elements) are known to those of skill in the art and are commercially available for generating the recombinant constructs.

As a representative but non-limiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and GEM 1 (Promega Biotech, Madison, Wis., USA). These pBR322 “backbone” sections are combined with an appropriate promoter and the structural sequence to be expressed. Other exemplary bacterial vectors include, for example, pBs, phagescript, PsiX174, pBluescript SK, pBs KS, pNH8a, pNH16a, pNH18a, pNH46a (Stratagene); pTrc99A, pKK223-3, pKK233-3, pDR540, and pRIT5 (Pharmacia). Following transformation of a suitable host strain and growth of the host strain to an appropriate cell density, the selected promoter is induced or derepressed by appropriate means (e.g., temperature shift or chemical induction) and cells are cultured for an additional period.

Mammalian expression vectors comprise an origin of replication, a suitable promoter, and also any necessary ribosome binding sites, polyadenylation site, splice donor and acceptor sites, transcriptional termination sequences, and 5′ flanking nontranscribed sequences. DNA sequences derived from the SV40 viral genome, for example, SV40 origin, early promoter, enhancer, splice, and polyadenylation sites may be used to provide the required nontranscribed genetic elements. Exemplary eukaryotic vectors include pWLneo, pSV2cat, pOG44, PXTI, pSG (Stratagene) pSVK3, pBPV, pMSG, and pSVL (Pharmacia).

Alternatively, a heterologous polynucleotide of the invention may be operably linked to an expression control sequence such as the pMT2 or pED expression vectors disclosed in Kaufman et al., Nucleic Acids Res. 19, 4485-4490 (1991), in order to produce the protein recombinantly. Many suitable expression control sequences are known in the art. General methods of expressing recombinant proteins are also known and are exemplified in R. Kaufman, Methods in Enzymology 185, 537-566 (1990). As defined herein, “operably linked” indicates that the isolated polynucleotide of the invention and an expression control sequence are situated within a vector or cell in such a way that the encoded protein is expressed by a host cell which has been transformed (transfected) with the ligated polynucleotide/expression control sequence.

For example, expression control sequences such as promoter regions can be selected from any desired gene. Bacterial promoters may include lacI, lacZ, T3, T7, gpt, lambda PR, and trc, and eukaryotic promoters include, for example, CMV immediate early, HSV thymidine kinase, early and late SV40, LTRs from retrovirus, and mouse metallothionein-I. Selection of the appropriate vector and promoter is well within the level of ordinary skill in the art. Generally, recombinant expression vectors will include origins of replication and selectable markers which permit identification and isolation of transformed host cells, e.g., the ampicillin resistance gene of E. coli and S. cereviisiae TRP1 gene, and a promoter derived from a highly expressed gene to direct transcription of a downstream structural sequence. Such promoters can be derived from operons encoding glycolytic enzymes such as 3-phosphoglycerate kinase (PGK), a-factor, acid phosphatase, or heat shock proteins, among others. The heterologous structural sequence is assembled in appropriate phase with translation initiation and termination sequences, and may include a leader sequence capable of directing secretion of translated protein into the periplasmic space or extracellular medium. Optionally, the heterologous sequence can encode a fusion protein including an amino terminal identification peptide imparting desired characteristics, e.g., stabilization or simplified purification of expressed recombinant product. Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and to, if desirable, provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice.

The present invention further provides host cells genetically engineered to contain the polynucleotides of the invention. The term “recombinant expression system” refers to host cells which have stably integrated a recombinant transcriptional unit into chromosomal DNA or carry the recombinant transcriptional unit extrachromosomally. Recombinant expression systems as defined herein will express heterologous polypeptides or proteins upon induction of the regulatory and secretory elements linked to the DNA segment or synthetic gene to be expressed. This term also means host cells which have stably integrated a recombinant genetic element or elements having a regulatory role in gene expression, for example, promoters or enhancers. Recombinant expression systems as defined herein will express polypeptides or proteins endogenous to the cell upon induction of the secretory signal sequence elements of the invention linked to the endogenous DNA segment or gene to be expressed. The cells can be prokaryotic or eukaryotic.

For example, such host cells may contain nucleic acids of the invention introduced into the host cell using known transformation, transfection or infection methods. The term “transformation” refers to the introduction of DNA into a suitable host cell so that the DNA is replicable, either as an extrachromosomal element, or by chromosomal integration. The term “transfection” as used herein refers to the taking up of an expression vector by a suitable host cell, whether or not any coding sequences are in fact expressed. The term “infection” refers to the introduction of nucleic acids into a suitable host cell by use of a virus or viral vector. Introduction of the recombinant construct into the host cell can be effected by calcium phosphate transfection, DEAE dextran mediated transfection, or electroporation (Davis, L. et al., Basic Methods in Molecular Biology (1986)).

The present invention still further provides host cells genetically engineered to express the polynucleotides of the invention, wherein such polynucleotides are in operative association with a regulatory sequence heterologous to the host cell which drives expression of the polynucleotides in the cell. The host cell can be a higher eukaryotic host cell, such as a mammalian cell, a lower eukaryotic host cell, such as a yeast cell, or the host cell can be a prokaryotic cell, such as a bacterial cell.

Any host/vector system can be used to express one or more of the polynucleotides of the present invention. Mature proteins can be expressed in mammalian cells, yeast, bacteria, or other cells under the control of appropriate promoters. Appropriate cloning and expression vectors for use with prokaryotic and eukaryotic hosts are described by Sambrook, et al., in Molecular Cloning: A Laboratory Manual, Second Edition, Cold Spring Harbor, N.Y. (1989), the disclosure of which is hereby incorporated by reference.

A number of types of cells may act as suitable host cells for expression of the protein. Mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells; mouse L cells, BHK, HL-60, U937, HaK or Jurkat cells. Also contemplated for use as host cells for expressing the chimeric polypeptide are insect Sf9 cells.

Alternatively, it may be possible to produce the protein in lower eukaryotes such as yeast or dictyostelium, in prokaryotes such as bacteria, or in viral systems. Potentially suitable yeast strains include Saccharomyces cerevisiae, Schizosaccharomyces pombe, Kluyveromyces strains, Candida, or any yeast strain capable of expressing heterologous proteins. Suitable fungal cell types include Dictyostelium discoideum, Dictyostelium polycephalum, Dictyostelium irregularis, Dictyostelium rhizopodium, Dictyostelium rhizopodium, Dictyostelium coeruleo-stipes, Dictyostelium lavandulum, Dictyostelium lavandulum, Dictyostelium vinaceo-fuscum, Dictyostelium deminutivum, Dictyostelium rosarium, Dictyostelium mucoroides, Dictyostelium dimigraformum, Dictyostelium laterosorum, Dictyostelium discoideum, and Dictyostelium purpureum. Suitable bacterial strains include Escherichia coli, Bacillus subtilis, Salmonella typhimurium, or any bacterial strain capable of expressing heterologous proteins. If the protein is made in yeast or bacteria, it may be necessary to modify the protein produced therein, for example by phosphorylation or glycosylation of the appropriate sites, in order to obtain the functional protein. Such covalent attachments may be accomplished using known chemical or enzymatic methods. Viral expression systems may also be used to generate the chimeric polypeptide, specifically contemplated include adenovirus, retrovirus, bacculovirus (as described in Summers and Smith, Texas Agricultural Experiment Station Bulletin No. 1555. 1987), and viral bacteriophages such as M13 or λ phage.

In another embodiment of the present invention, cells and tissues may be engineered to express a secreted protein from an endogenous gene, which is not normally expressed or expressed at a relatively low level, wherein sequences of the endogenous gene supplemented with and/or replaced by a polynucleotide encoding a secretory signal polypeptide of the invention using homologous recombination. As described herein, gene targeting can be used to introduce the secretory signal-encoding polynucleotide by simple insertion of the secretory signal sequence, and may also include placing the gene under the control of a regulatory sequence, e.g., inserting a new promoter or enhancer or both upstream of a gene. Alternatively, the targeting event may replace an existing element; for example, a transmembrane protein anchoring domain can be replaced by a secretory signal sequence that has different protein anchoring properties than the naturally occurring elements. Here, the naturally occurring sequences are deleted and new sequences are added. In all cases, the identification of the targeting event may be facilitated by the use of one or more selectable marker genes that are contiguous with the targeting DNA, allowing for the selection of cells in which the exogenous DNA has integrated into the host cell genome. The identification of the targeting event may also be facilitated by the use of one or more marker genes exhibiting the property of negative selection, such that the negatively selectable marker is linked to the exogenous DNA, but configured such that the negatively selectable marker flanks the targeting sequence, and such that a correct homologous recombination event with sequences in the host cell genome does not result in the stable integration of the negatively selectable marker. Markers useful for this purpose include the Herpes Simplex Virus thymidine kinase (TK) gene or the bacterial xanthine-guanine phosphoribosyl-transferase (gpt) gene.

The gene targeting or gene activation techniques which can be used in accordance with this aspect of the invention are more particularly described in U.S. Pat. No. 5,272,071 to Chappel; U.S. Pat. No. 5,578,461 to Sherwin et al.; International Application No. PCT/US92/09627 (WO93/09222) by Selden et al.; and International Application No. PCT/US90/06436 (WO91/06667) by Skoultchi et al., each of which is incorporated by reference herein in its entirety.

The invention further provides methods for production of a secreted polypeptide comprising the step of culturing transformed host cells under culture conditions suitable for growth and which permit the expression and secretion of the heterologous polypeptide. The method for expression of the secreted polypeptide can also comprise the step of cleaving the secretory polypeptide from the heterologous polypeptide at the peptide cleavage site. Cleavage in this instance refers to the separation of the first, heterologous polypeptide from the secretory signal polypeptide wherein the cleavage results in release of a biologically functional heterologous polypeptide. The cleavage of the chimeric nucleic acid can be carried out extracellularly or intracellularly. “Extracellular cleavage” is performed by administration of a cleaving agent after isolation of the secreted polypeptide from the host cell by specific enzymatic or chemical cleavage reaction. “Intracellular cleavage” is carried out by the administration of a cleaving agent directly to the host cells producing the secreted polypeptide, such that the host cell secretes an isolatable first polypeptide in to the cell media. Alternatively, the endogenous activity of furin present in the endoplasmic reticulum can be used to mediate peptide cleavage.

Polypeptide cleavage can be carried out using either chemical or enzymatic cleavage methods (Unit 16.4.5, Current Protocols in Molecular Biology, John Wiley and Sons, 2001). To carry out a specific peptide cleavage reaction for the chimeric polynucleotide of the invention, the amino acid sequence including the site of enzymatic or chemical cleavage is inserted into the chimeric polypeptide between the heterologous polypeptide coding region and the secretory signal by recombinant polynucleotide modification. Exemplary chemical cleavage processes include cyanogens bromide cleavage after methionine residues or hydroxylamine cleavage between Asparagine and Glycine residues. More specific cleavage is mediated via enzymatic digestion at particular amino acid residues such as serine or threonine. Exemplary enzymatic peptide cleavage can be carried out by Factor Xa (Ile-Glu-(or Asp)-Gly-Arg↓), enterokinase (Asp-Asp-Asp-Asp-Lys↓), thrombin (Leu-Val-Pro-Arg↓Gly-Ser) or furin (RSKR↓, or RKKR↓) proteases.

In another technique, the cre-lox system can be used to remove the secretory signal sequence tag. The system consists of 34 base pair lox sequences that are recognized by the bacterial cre recombinase gene. The lox sites are present in the DNA in an appropriate orientation, and DNA flanked by the lox sites is excised by the cre recombinase, resulting in the deletion of all DNA sequences between the lox repeats except for one remaining copy of the lox sequence. Using standard recombination techniques, it is possible to delete the targeted secretory signal sequence DNA. Transient expression (by electroporation of a suicide plasmid containing the cre gene should result in efficient elimination of the lox flanked marker, resulting in a first nucleotide sequence without the attached secretory tag.

The chimeric polypeptide can be isolated using various techniques well-known in the art. One skilled in the art can readily follow known methods for isolating polypeptides and proteins in order to obtain one of the isolated polypeptides or proteins of the present invention. These include, but are not limited to, immunochromatography, HPLC, size-exclusion chromatography, ion-exchange chromatography, and immuno-affinity chromatography. See, e.g., Scopes, Protein Purification: Principles and Practice, Springer-Verlag (1994); Sambrook, et al., in Molecular Cloning: A Laboratory Manual; Ausubel et al., Current Protocols in Molecular Biology. Isolation of the chimeric polypeptide from the cell culture media can be facilitated using antibodies directed against either the first, heterologous polypeptide or the secretory signal polypeptide or fragments thereof that will be readily recognized by the antibody, and then isolated via immunoprecipitation methods and separation via SDS-polyacrylamide gel electrophoresis (PAGE). Alternatively, the chimeric protein can be isolated via binding of the polypeptide-specific antibody to the polypeptide-secretory tag chimera and subsequent binding of the antibody to protein-A or protein-G Sepharose columns, and elution of the protein from the column.

The purification of the protein may also include an affinity column containing agents which will bind to the protein; one or more column steps over such affinity resins as concanavalin A-agarose, heparin-toyopearlJ or Cibacrom blue 3GA SepharoseJ; one or more steps involving hydrophobic interaction chromatography using such resins as phenyl ether, butyl ether, or propyl ether; or immunoaffinity chromatography.

Alternatively, the protein of the invention may also be expressed in a form which will facilitate purification. For example, it may be expressed as a fusion protein, such as those of maltose binding protein (MBP), glutathione-S-transferase (GST) or thioredoxin (TRX), or as a His tag. Kits for expression and purification of such fusion proteins are commercially available from New England BioLab (Beverly, Mass.), Pharmacia (Piscataway, N.J.) and Invitrogen, respectively. The protein can also be tagged with an epitope and subsequently purified by using a specific antibody directed to such epitope. One such epitope (“FLAG®”) is commercially available from Kodak (New Haven, Conn.).

Finally, one or more reverse-phase high performance liquid chromatography (RP-HPLC) steps employing hydrophobic RP-HPLC media, e.g., silica gel having pendant methyl or other aliphatic groups, can be employed to further purify the protein. Some or all of the foregoing purification steps, in various combinations, can also be employed to provide a substantially homogeneous isolated recombinant protein. The protein thus purified is substantially free of other mammalian proteins and is defined in accordance with the present invention as an “isolated protein.”

The present invention is illustrated in the following examples. Upon consideration of the present disclosure, one of skill in the art will appreciate that many other embodiments and variations may be made in the scope of the present invention. Accordingly, it is intended that the broader aspects of the present invention not be limited to the disclosure of the following examples. The present invention is not to be limited in scope by the exemplified embodiments which are intended as illustrations of single aspects of the invention, and compositions and methods which are functionally equivalent are within the scope of the invention. Indeed, numerous modifications and variations in the practice of the invention are expected to occur to those skilled in the art upon consideration of the present preferred embodiments. Consequently, the only limitations which should be placed upon the scope of the invention are those which appear in the appended claims.

All references cited within the body of the instant specification are hereby incorporated by reference in their entirety.

### EXAMPLE 1

Detection of Thy-1 Fused to Truncated gD at the Cell Surface

The polypeptide gDww63 is a truncated form of the 394 amino acid herpes simplex virus I glycoprotein D (GENBANK ACCESSION #Q69091) comprising residues 1-337 of SEQ. ID NO.: 2. The gDww63 peptide truncate lacks the glycoprotein D hydrophilic and charged cytoplasmic domain, and displays all the features of a GPI-anchored protein. Previous work has shown that the C-terminal region of gDww63, comprising amino acid residues 234-337 of SEQ ID NO.: 2 and designated gDL (SEQ ID NO: 6), is able to induce transfer of a GPI-anchor onto a reporter molecule (Beghdadi-Rais et al., J. Cell Sci. 105:831-40. 1993). In order to determine if the gDL peptide is efficiently processed and modified by a GPI anchor in the same manner as the full length gD protein and the gDww63 peptide, a fusion protein comprising the T cell marker protein Thy-1 and the gDL peptide was prepared. Expression and post-translational addition of the GPI anchor onto the Thy-1/gDL fusion protein was assessed in P815 mouse mastocytoma cells, together with a second fusion protein comprising the gDww63 peptide and Thy-1, designated Thy-1/gDww63 (Beghdadi-Rais et al., supra).

A polynucleotide encoding the 337 amino acid gDww63 peptide was prepared by replacing the codon for tryptophan at position 338 with a stop codon, thereby deleting the cytoplasmic tail of the wild type HSV gD-1 protein. The Thy-1/gDww63 expression vector was prepared as previously described (Beghdadi-Rais et al., supra). A polynucleotide encoding the Thy-1/gDL fusion protein, composed of the ectoplasmic segment of the Thy-1 antigen (Genbank Accession No. M11160, amino acids 1-114, SEQ. ID NO. 4) and of the last 103 residues of gDww63 (amino acids 234-337, SEQ ID NO. 2 and SEQ. ID NO.: 6), was prepared by subcloning the AccI-BamHI fragment from pRSVgDww63 construct into the EcoRV site of pTSV2μNF13, which was previously obtained after subcloning the 170 base pair SacI-XhoI fragment of Thy-1 into the pTSV2μ plasmid (Wilson et al., J. Cell Science 96; 143-149. 1990). The expression constructs were sequenced either directly using SP6 and T7 primers for pCR3 derived constructs or after subcloning of the region of interest into pGEM-1. DNA sequences are determined using the dideoxy chain termnination method (Sequenase Kit, Pharmacia) on an automated sequencer. Cell transfection was carried out using standard procedures.

For detection of fusion protein products of the expression constructs at the cell surface, cells were incubated 1 hour at 4° C. with a 1:1000 dilution of rabbit antiserum to mouse Thy-1 antigen (MacDonald et al., Eur. J. Immunol. 15: 495-501. 1985) or a 1:2500 dilution of rabbit antiserum to herpes simplex gD-1 glycoprotein (Isola et al., J. Virol. 63: 2325-34. 1989) and subsequently washed by centrifugation through a cushion of fetal calf serum (FCS). Cells were spread on a slide with a cytospin centrifuge (Shandon Southern) and fixed 4 minutes in acetone at −20° C. Endogenous peroxidase was blocked by incubating the cells for 10 min in phosphate buffer B (20 mM NaH₂PO₄, 80 mM Na2HPO4, 1.5 M NaCl) containing 0.3% H₂O₂ and 0.1% NaN₃. For internal labeling, the same protocol is followed, however, incubation with the polyclonal rabbit anti-Thy-1 (R191) (MacDonald et al., Eur. J. Immunol. 15:495-501. 1985) or anti-gD-1 (R45) (Isola et al., J. Virol. 63:2325-34. 1989) antibody was performed after the endogenous peroxidase blocking step. Unbound antibody was washed from the cells with phosphate buffer C (100 mM NaH₂PO₄, 400 mM K₂HPO₄, 1.2 M NaCl, 0.49 mM Thiomersal) and biotinylated goat anti-rabbit (Vector Laboratories, Inc, Burlingame, Calif.) added at 25 μg/ml. After 30 min, slides were washed and incubated for 15 min with avidin-peroxidase (Vector Laboratories).

Immunochemical staining with either anti-Thy-1 or anti-gd antibodies demonstrated that the Thy-1/gDL glycoprotein was detected on the cell surface as well as intracellularly in P815 mastocytoma cells. No signal was observed in non-transfected cells with either antisera. Similarly, when the Thy-1/gDww63 fusion protein was expressed in same P815 cells, it is detected both intracellularly and at the cell surface. Although these results demonstrated that the Thy-1/gDL fusion protein can be expresses at the cell surface, they do not indicate the exact mechanism of attachment to the cell membrane, i.e. GPI-anchor or transmembrane peptide.

### EXAMPLE 2

Thy-1/gDL Molecules Attached via GPI Anchor

In order to determine if the surface-expressed Thy-1/gDL fusion protein was anchored in the plasma membrane via a GPI structure, the following assay was carried out using the transfected P815 cells expressing the Thy-1/gDL fusion protein.

Lactoperoxidase-catalyzed cell surface iodination was carried out as described (Hubbard et al., J. Biol. Chem. 64:438-60. 1975), after which phospholipase C treatment was performed as follows. Approximately 2×10⁷ transfected P815 cells were lysed on ice in 1 ml of 1% Triton X-114 in presence of 55 μM leupeptin, 40 μM pepstatin and 7 μM antipain and subjected to phase separation at 32° C. (Bordier et al., J. Biol. Chem. 256:1604-07. 1981). The detergent phase containing amphiphilic proteins was extracted twice by the addition of a ten-fold volume of NaCl/Tris (0.15 M NaCl, 0.1 M Tris, pH 7.4) with 1 mM EDTA and 0.06% Triton X-114, followed by phase separation to remove remaining water-soluble proteins. Washed detergent phases were adjusted to 0.1 M Tris-HCl pH 7.4, 0.05 M NaCl, 1 mM EDTA, and diluted to a final Triton X-114 concentration of 3-4%. Phosphatidylinositol-specific phospholipase C from Bacillus cereus (1 unit) (Roche/Boehringer-Mannheim) was added, incubation carried out for 60 min at 37° C., and aqueous and detergent phases separated after the addition of NaCl/Tris (1 mM EDTA and 0.06% Triton X-114). Detergent and aqueous phases were extracted by two cycles of Triton X-114 phase separation and diluted in 1.2 ml of buffer containing 25 mM Tris-HCl pH 8.2, 50 mM NaCl, 0.5% Nonidet P40, 0.5% deoxycholic acid and 0.01% NaN₃ for immunoprecipitation.

Detergent and aqueous phases were immunoprecipitated with rabbit antisera to mouse Thy-1 antigen (MacDonald et al., Eur. J. Immunol. 15:495-501. 1985) or to herpes simplex gD-1 glycoprotein (Isola et al., J. Virol. 63:2325-34. 1989), followed by adsorption onto protein A-Sepharose beads according to previously described procedures (Beghdadi-Rais et al., supra). After 12.5% SDS PAGE, autoradiography of the dried gel was performed at −70° C. using KODAK XAR-5 film in the presence of an intensifying screen.

Signals were observed at the expected molecular weight in the detergent phases (32-36 kDa, due to glycosylation) indicating that PI-PLC treatment resulted in the transfer of Thy-1/gDL molecules from the detergent into the aqueous phase, confirming that the gDL C-terminal region of gDww63 possesses all the elements necessary to signal GPI anchor addition. No signal was observed in the aqueous phase when the PI-PLC was omitted from the reaction. Results indicate that gDww63 and Thy-1/gDL are attached to the membrane via a glycolipid structure. In a control GPI-deficient cell line, no surface expression of GPI anchored molecules was observed.

### EXAMPLE 3

Expression of gDww63 and Thy-1/gDL In GPI-Deficient Cell Lines

In an attempt to assess whether the C-terminal segment of gDww63 functions strictly as a true GPI anchor addition signal, the expression of chimeric gDL constructs in a GPI deficient cell line was analyzed.

To generate a GPI-deficient cell line, chemical mutagenesis was performed on the P815-620 cell line which expresses Thy-1 in a GPI-anchored form (Déglon, 1992, unpublished results). Approximately 4×10⁷ P815-620 cells, previously cloned and characterized as Thy-1⁺ by cell surface analysis, were cultured for 15 hours in 120 ml of DMEM medium in the presence of 200 μg/ml ethylmethanesulfonate (Sigma). The cells were washed twice and suspended in fresh medium. Cells previously incubated with a rabbit antiserum to mouse Thy-1 antigen (MacDonald et al., Eur. J. Immunol. 15:495-501. 1985) were transferred to dishes coated with anti-rabbit IgG. Non-adhering cells represented the mutagenized GPI deficient population and these cells were analyzed by fluorescence activated cell sorting (FACS) using standard procedures.

For FACS assay, approximately 1×10⁶ cells were suspended in 300 μl of medium containing 5% fetal calf serum. Cell suspensions were incubated 45 min at 4° C. with a 1:2000 dilution of rabbit antisera to mouse Thy-1 antigen (MacDonald et al., Eur. J. Immunol. 15:495-501. 1985). Cells were washed by centrifugation through a cushion of fetal calf serum (FCS), and resuspended in 110 μl medium containing 5% FCS and 10 μl commercial biotin-conjugated donkey anti-rabbit IgG (Amersham). After a 45 min incubation at 4° C., the cells were washed and resuspended in 100 μl medium containing 5% FCS. Finally, cells were incubated for 1 hour at 4° C. with 10 μl of fluorescein-conjugated streptavidin (Amersham), washed, and analyzed on a fluorescence-activated cell sorter (FACS II System, Becton Dickinson, Franklin Lakes, N.J.).

Twelve clones were analyzed and shown to be Thy-1 negative indicated GPI deficiency and a single clone designated P815-C9 was used for further studies. As a control [³⁵S]methionine labeling confirmed that Thy-1 was synthesized in the P815-C9 cell line.

### EXAMPLE 4

Analysis of Labeled Lipids in a GPI-Deficient Cell Line

In order to confirm the nature of the mutation in the P815-C9 cell line, the following experiments were carried out. In brief, [³H]-myo-inositol labeled lipids from wild type cells, from various GPI deficient lines, and from the P815-C9 cell line were compared by thin layer chromatography to identify the genetic defect in the P815-C9 cells.

Parental cell lines of S1A, P815-444, and P815-620, as well as the mutant lines S1 A/B, BW5147/A, TIM1/C, BW5147/E, EL4/F, S49/H, and P815-C9 were labeled as follows. Cells were washed and suspended in inositol-free medium at 10⁶ cells/m (with serum dialyzed against Hank's balanced salt solution); [³H] myo-inositol (Amersham) was added as an aqueous solution and cells were incubated at 37° C. After labeling, cells were washed, and labeled lipids were extracted twice for 45 min using CHCl₃/CH₃OH/H₂O (10:10:3). The lipid extracts were dried under nitrogen and are stored in butanol or CHCl₃/CH₃OH/H₂O (10:10:3) at −20° C. Lipids were desalted by partitioning between butanol and water as described (Krakow et al., J. Biol. Chem. 261:12147-53. 1986). Ascending thin-layer chromatography (TLC) was performed on a 0.2-mm-thick Silica Gel 60 plates (Merck) using CHCl₃/CH₃OH/0.25% KCL in water (55:45:10) as solvent. The developed TLC plate was sprayed with EN3HANCE (DuPont-New England Nuclear, Boston, Mass.) and the fluorogram was obtained using X-OMAT film (Kodak) exposed for 6 days at −80° C.

TLC analysis of the position of the origin (ori), the phosphatidylinositol (PI) moiety, and of the mature glycolipid anchor (CP) were measured for each cell line. The TLC migration pattern of the [³H]-myo-inositol labeled lipids of the P815-C9 cells was different from the pattern obtained from wild type cells S1A T lymphoma cells, P815-444 and P815-620 cells, as evidenced by the absence of the mature GPI anchor (CP) in the P815-C9 cell line. The P815-C9 pattern was also different from the pattern of the mutant line S1A/B, BW5147/E and EL4/F lines, but was similar to the pattern observed for the complement classes A, C and H which synthesize normal lipids, but lack the GPI anchor precursor, CP.

These results indicated/suggested that the P815-C9 cell line efficiently produces lipid moieties but is defective in the GPI-anchor addition mechanism.

### EXAMPLE 5

Identification of Genetic Defect in P815-C9 GPI-Deficient Cell Line

In an attempt to more precisely define the genetic defect in the P815-C9 cell line, transfection experiments were performed in an attempt to correct the GPI deficiency.

In a first experiment, expression of Thy-1/gDL in P185-C9 cells was tested to assess the presence of the fusion protein at the cell surface which would determine if Thy-1/gDL interacts with the membrane via a peptidic segment rather than with a GPI-anchor. In this assay, transfected cells were treated with or without PI-PLC and examined by FACS.

Cell surface fluorescent intensity measurement showed that PI-PLC treatment had no effect on Thy-1/gDL fluorescence intensity confirming that Thy-1/gDL was attached to the surface via a peptidic segment rather than a GPI anchor. No fluorescence was observed in non-transfected cells.

In a second experiment, cell surface expression was examined using the same mutant cell line co-transfected with a gene encoding PIG-A.

An expression construct designated pSV2-PIG-A was obtained by subcloning a PCR-amplified cDNA fragment of the PIG-A gene into the XhoI and BamHI restriction sites of vector pSVL-pW2 derived from pSVL and previously described (Breathnach et al., Nuc. Acids Res. 11:7119-36. 1983). The oligonucleotide primers (SEQ ID NOs: 9 and 10) used for the PCR were deduced from the published PIG-A cDNA sequence (Miyata et al., Science 259:1318-20. 1993) (SEQ. ID NO: 7).

| | |
|---|---|
| 5′-GTCCTCGAGTCTGCAGCATGGCCTGT-3′ | (SEQ. ID NO: 9) |
| 5′-TAGGGATCCTTCTACCTGGTTTC-3′ | (SEQ. ID NO: 10) |

The plasmids pSV2-neo and pSV2-hygro used in the co-transfection experiments have been described elsewhere (Southern et al., J. Mol. Appl. Gene. 1:327-41. 1982), (Fasel et al., Immnunogenetics 35:126-30. 1992).

Expression of the PIG-A gene in P815-C9 cells was shown to restore cell surface expression of a PI-PLC-sensitive fusion protein and demonstrated that the P815-C9 cells belong to the complementation class A, which are defective in transfer of GIcNAc to PI. PI-PLC treatment reduced the amount of specific cell surface fluorescence of either protein by 50%. This value is approximately the level found with Thy-1/gDL expressing P815-C9 cells observed in the first experiment. These results confirmed that expressing the PIG-A gene in the P815-C9 cells restored expression of GPI-anchored Thy-1/gDL at the cell surface. Interestingly, a higher level of fluorescence intensity was observed with the anti-Thy-1 antibody than with the anti-gD-1 antibody, and this difference can be explained by the fact that the complementation of the P815-C9 cells by the PIG-A gene not only restored cell surface expression of the chimeric Thy-1/gDL molecule but also of wild type Thy-1 molecules which are retained intracellularly in the P815-C9 cells. No expression of Thy-1/gDL was observed in non-transfected cells either with anti-Thy-1 antibody or with an anti-gD antibody. Experiments conducted with the gDww63 construct elicited similar results (such as the expression of PI-PLC-resistant gDww63 protein at the surface of the P815-C9 cells), demonstrating that the C-terminal gDL 103 amino acids of the gDww63 polypeptide can anchor a polypeptide in the membrane via a peptidic sequence and a GPI anchor. Thus, both types of anchors (TM- and GPI-anchor) are present on the gD chimeric molecules. In order to confirm these results, a third experiment was carried out as follows.

Mouse L cell fibroblasts are also GPI anchor deficient and release GPI-anchor-containing molecules into the culture medium (Ferguson et al., Science 239:753-9. 1988), (Edelman et al., Pro. Natl. Acad. Sci. USA 84:8502-06. 1987; Camerini et al., Nature 342:78-82. 1989; Stroynowski et al., Cell 50:759-68. 1987; Fasel et al., Cell Biol. Intl. Reports 15: 1051-1064. 1991) due to a genetic lesion in the PIG-H gene (Singh et al., Mol. Cell Biol. 11.2362-74. 1991). Exponentially growing L cells, 80% confluent, were co-transfected by the calcium phosphate method (Chen et al. Mol. Cell Biol. 7:2745-52. 1987) with 10 μg of pRSVgDww63 and 1 μg of pSV2-hygro DNA. The transfection was carried out for 15 hours at 37° C. under 5% CO₂. After transfection, the cells were washed with medium and incubated for 24 hours. Selection was achieved in the presence of 210 μg/ml hygromycin B (Calbiochem). Selected cells were then incubated in the presence and absence of PI-PLC.

Flow cytometric analysis of L cells stably transfected with Thy-1/gDL indicated that the fusion protein was expressed at the cell surface and were resistant to PI-PLC treatment, confirming that Thy-1/gDL attached to the cell surface of the GPI-deficient lines via a TM-anchor. These results again confirm that membrane anchoring of the proteins gDww63 and Thy-1/gDL occurs via both a peptidic transmembrane segment and a GPI-anchor.

### EXAMPLE 6

Release of gD Into the Cytoplasm Due to a Distinct Segment of gD, gDS

In parallel to the study concerning the mode of anchoring of Thy-1/gDL and of gDww63, assays were carried out to determine if these proteins could also be released into the medium. The release of Thy-1/gDL and gDww63 into the culture medium could be due to the low hydrophobicity of the gDww63 C-terminus sequence. Alternatively, it is possible that a domain present in gD selectively influences the interaction with the membrane. To test this hypothesis, various constructs were engineered based on the assumption of the presence of a gD specific segment which would influence the anchoring in the membrane.

The C-terminal 103 amino acid sequence of gDww63 was divided into two segments. The first amino acid sequence, gDS, corresponds to the 60 amino acid residues 234-294 in gDww63 (SEQ. ID Nos.: 29 and 30) and the second region (gD 10) corresponds to the last 43 amino acids of gDww63 (residues 294-337). To obtain the Thy-1/gDS-encoding construct, Thy-1/gDL was digested with BstXI/HindIII and the 400 bp fragment was replaced by a PCR product of the same size containing a stop codon, TAG, after the amino acid W294. The oligonucleotide primers used in this PCR are set out below as SEQ ID NOs: 11 and 12.

| | |
|---|---|
| 5′-CCGCAAATCCCACCAAACTGGTAG- | (SEQ. ID NO:11) |
| CTCGAGTCGATCCAGGACGAA-3′ | |
| 5′-ATAAGCTTGCCGAAAAAGCTGTGG-3′ | (SEQ. ID NO:12) |

To prepare a Thy-1/gD10 expression vector, the DNA fragment corresponding to gD amino acids P294 to Y337 (nucleotides 871-1011 in SEQ ID NO: 1) as well as the downstream 260 nucleotides of Thy-1/gDL were amplified by PCR, digested with EcoRV and HindIII, blunt-ended using T4 DNA polymerase and inserted into the EcoRV site of Thy-1/NF13 (Beghdadi-Rais et al., J. Cell Sci. 105: 831-40. 1993). The oligonucleotide primers used in this PCR are set out below.

(SEQ. ID NO:13)

5′-GTGGCGCCGGATATCCCCACCAAACTGGCAC-3′

(SEQ. ID NO:14)

5′-ATAAGCTTGCCGAAAAAGCTGTGG-3′

HEK293T cells, cultured in Dulbecco's modified Eagle's medium with Glutamax-1 (Gibco BRL) supplemented with 5% FCS and 10 μg/ml gentamycin, were transfected by the described calcium phosphate method (Van Pel et al., Som. Cell. Mol. Genet. 11:465-75. 1985) with 10 μg of the desired truncated gD construct. The transfection was carried out for approximately 18 hours at 37° C. under 5% CO₂. Following transfection, the cells were washed twice with medium and incubated for 48 or 72 hours. The cells were harvested by 5 minutes of centrifugation and the cells suspended in sample buffer and boiled before electrophoresis. The medium was cleared by a second centrifugation of 5 minutes and protein in the medium precipitated 1 hour in 10% trichloroacetic acid (TCA) at 4° C. After one washing step with acetone, 1×10⁵ cells or 0.5 ml of medium (TCA precipitated) was suspended in 10 μl of sample buffer and heated 5 minutes at 95° C. before being loaded on 10% or 12% SDS polyacrylamide gel (PAGE) and subjected to electrophoresis. The gel was subsequently stained 20 minutes in a solution of Coomassie Brilliant Blue R250 in 10% acetic acid and 25% isopropanol. The gel was destained 3 hours in 10% acetic acid and 25% isopropanol, and dried under vacuum at 55° C.

Immunoblot analysis of cell culture supernatant from HEK293T cells transfected with either the Thy-1, Thy-1/gDL, Thy-1/gDS or Thy-1/gD10 constructs demonstrated that only cells transfected with the Thy-1/gDL and Thy-1/gDS constructs showed detectable levels of the fusion protein in the culture medium as detected by rabbit anti-Thy-1 antiserum. When Thy-1 was attached to the cell membrane via its GPI-anchor, no signal was detected in the culture medium. Similarly, no Thy-1 signal was detected in the culture medium of cells transfected with the Thy-1/gD10 fusion construct. Considering that Thy-1/gDL and Thy-1/gD10 differ only by the presence of 60 amino acids corresponding to the gDS sequence in the gDL segment and not by their hydrophobic segment, it can be concluded that the release of the fusion molecule is not due to a defect in addition of the GPI-anchor or to a different hydrophobicity of the C-terminal segment.

These results confirm that the presence of gDS not only has a strong influence on the level of secreted molecules but is an essential segment in the gDL fragment necessary to obtain secretion by this polypeptide.

### EXAMPLE 7

Increased Secretion of the Adhesion Molecule JAM-2 by gDS

In order to assess the ability of the gDS polypeptide fragment to regulate the secretion of proteins, gDS polypeptide-encoding constructs were created to express the gDS fragment fused to other proteins characteristically found as membrane-bound polypeptides or proteins requiring chaperone proteins to arrive at the cell surface.

JAM-2 is a human adhesion membrane protein expressed at the surface of endothelial cells and is normally not released in the culture medium (Aurrand-Lions, et al. J. Biol. Chem. 276: 2733-2741. 2001). Its ectoplasmic domain is composed of 238 amino acids. To alter the level of secretion of JAM-2, an expression construct was engineered in which the gDS segment was expressed as a fusion protein with the C-terminus of the ectoplasmic domain of JAM-2. To obtain plasmid pCR3/gDS encoding the gDS sequence, the nucleotide sequence encoding gDww63 corresponding to amino acids Y234 to W294 of gDww63 was amplified by PCR using oligos primers set out below.

(SEQ. ID NO: 15)

5′-AGGATATCTACAGCTTGAAGATCGCCGGG-3′

(SEQ. ID NO: 16)

5′-TGCTCGAGTCACCAGTTTGGTGGGATTTGCGG-3′

The amplified fragment was then subcloned into the EcoRV and XhoI restriction sites of pCR3 (Invitrogen, Carlsbad, Calif.).

To obtain the plasmid pCR3/JAM-2 encoding JAM-2, a PCR fragment corresponding to amino acids 1-238 of JAM-2 (SEQ. ID NO: 18), with a stop codon after Y238 and 10 nucleotides of murine JAM-3 5′ untranslated region (UTR) before the ATG, was amplified and inserted into the HindIII and EcoRV restriction sites of the pCR3 vector. Plasmid PCR3/JAM-2/gDS encoding the JAM-2/gDS fusion protein was obtained by inserting into the HindIII and EcoRV restriction sites of pCR3/gDS a PCR amplification fragment encoding the JAM-2 sequence without the transmembrane domain of the protein (amino acids 1-238) and including the 10 upstream nucleotides of murine JAM-3. The oligonucleotide primers used in the PCR reaction are set out below.

(SEQ. ID NO: 19)
5′-GACTGTAAGCTTGCCCGCGTAGATGGCGCTGAG
GCGGCCAC-3′
(SEQ. ID NO: 20)

5′-CGTCAAGATATCATAGACTTCCATCTCCTGCTCC-3′

The JAM-2/gDS construct was transfected into HEK293T cells as previously outlined and the cell culture supernatant of HEK293T transfected cells was analyzed by simple staining of gel-resolved protein or by immunoblotting and compared to non-transfected cells.

By Coomassie blue staining of the gel, a specific band was visible only in the culture medium of cells transfected with the JAM-2/gDS construct. A signal corresponding to the expected molecular mass of JAM-2/gDS was also detected by immunoblotting using an anti-gD antibody. No signal was detected in non-transfected cells or cells transfected with truncated JAM-2 alone. These results confirm that the presence of gDS has a strong influence on the level of secreted molecules.

This JAM-2/gDS construct could be used to inhibit the transmigration of lymphocytes and platelet binding. JAM-2/gDS could also be used as a therapeutic in the treatment of transplantation rejection and in minimizing inflammation in many diseases, including rheumatoid arthritis.

### EXAMPLE 8

Secretion of a Rubella Viral Antigen Fused to gDS

In view of the results discussed above, additional experiments were carried out to assess the ability of the specific gD sequences to induce secretion of proteins which are not normally secreted.

Rubella virus envelope glycoproteins E1 and E2 are targeted to the Golgi apparatus as heterodimers and E1 requires heterodimerization with E2 in order to reach the Golgi. It has previously been shown that addition of a GPI anchor to the E1 protein targets E1 to the plasma membrane and that co-expression of a mammalian GPI-phospholipase E results in the release of E1 into the medium (Bernasconi et al., J. Cell Sci. 109:1195-201. 1996), indicating that the E1 requirement for E2 dimerization to reach the cell surface can be overcome by the addition of a GPI-anchor. To investigate the role of gDS on the release of this protein into the medium, the gDS sequence was fused to the E1 rubella virus antigen.

The construct pCB6-E1ass TMCT-6His has been described previously (Bernasconi et al., supra). The construct pCR3/LP/E1/gDS encoding a gDS/E1 fusion proteins was derived from pCR3/gDS as follows. In a first step, the nucleotide sequence corresponding to the signal sequence of Thy-1 (SEQ ID NO. 21) plus 10 nucleotides of 5′ UTR were amplified and inserted into the HindIII and BamHI restriction sites of pCR3/gDS. The oligonucleotide primers used in the PCR reaction are set out below.

(SEQ. ID NO:23)

5′-CAAGTCAAGCTTGGCACCATGAACCCAGCCAT-3′

(SEQ. ID NO:24)

5′-ATGAACGGATCCCCTCTAGAGGTCACCTTCTGAAATCGGG-3′

In a second step, the nucleotide sequence encoding rubella virus E1 (Genbank Accession No. X05259) amino acid residues 1 to 412 (SEQ. ID NO: 26) without its signal sequence and its transmembrane and cytoplasmic domains was amplified by PCR using pCB6-E1ass-GPI (Bernasconi et al., supra) as template and the following oligo primers.

(SEQ. ID NO: 27)

5′-ATGCAGATCTCGAGGAGGCTTTCACCTACCTC-3′

(SEQ. ID NO: 28)

5′-TACCGATATCATGACCCGCGCTCGCGCGAT-3′

This amplified fragment was digested by the restriction enzymes Bg1II and EcoRV and inserted into the BamHI and EcoRV restriction sites of the construct described above. The E1/gDS construct was transiently expressed into HEK293T cells and the presence of E1 in the supernatant was analyzed by immunoblotting as outlined previously.

Results indicated that the 62 kDa chimeric protein was detected intracellularly and in the medium with anti-gD (R45) antibody or with anti-E1 antibodies. No E1 was detected in non-transfected cells either intracellularly or in the medium with anti-E1 or anti-gD antibodies. This result suggests that the gDS sequence can circumvent the ER block observed earlier with E1 protein. When the E1 was expressed without its transmembrane segment (construct E1 TM), E1 is not detected in the medium but is detected in the cellular pellet as a doublet with molecular masses of approximately 30 kDa. The difference in molecular mass between E1/gDS and E1 is due to the presence of 60 amino acids of gDS and the presence of one glycosylation site.

This result demonstrated that the gDS sequence has a dominant effect over the E1 pre-Golgi retention signal and requirement for a chaperone protein, and thus, can be used to obtain soluble polypeptides in large quantities.

### EXAMPLE 9

Fusion of gDS to gH of HSV-2

To further confirm the importance of gDS as a secretory signal peptide, gDS was fused to other viral membrane antigens which require a chaperone protein for transport to the cell surface.

It has previously been shown that dimerization of glycoproteins gH and gL of herpes simplex virus is necessary for correct folding and targeting of both glycoproteins to the cell surface (Dubin et al, J. Virology 69: 4564-68. 1995). In the absence of gL, gH is incompletely processed, improperly folded, and retained intracellularly. In view of these observations, a herpes chimeric molecule was generated by fusing the ectoplasmic domain of HSV-2 glycoprotein H (gH-2) to gDS.

The pCR3/gH-2/gDS expression construct was derived from pCR3/gDS described previously. The nucleotide sequence encoding amino acids 1 to 803 of gH-2 was amplified by PCR using the gH-2 coding region as template and the following oligonucleotide primer.

(SEQ. ID NO.: 66)

5′-TCGGTACCATGGGCCCCGGTCTGT-3′ and

(SEQ. ID NO.: 67)

5′-TGGAATTCCCGGGGGCGATGGTGGCGATG-3′.

The amplified gH-2 sequence was then inserted into the Asp718 and EcoRI restriction sites of pCR3/gDS and the resulting construct transfected into HEK293T cells. The presence of the encoded fusion proteins, either intracellularly or in the cell culture medium, was analyzed by immunoblotting using rabbit anti-gD-1 antiserum.

After electrophoresis, the gH-2 fusion protein migrating with the predicted molecular mass of 97 kDa was detected both intracellularly and in the extracellular medium of transiently transfected HEK293T cells. No gH-2 fusion protein signal was detected in non-transfected cells. A difference in the pattern of migration was observed between the intracellular species and forms released into the medium and could possibly be attributed to differences in glycosylation of the two species.

### EXAMPLE 10

Fusion of gDS to Proteins of Interest to Alter Cell Surface Expression

It is often useful to be able to modulate, either stimulate or diminish, the expression of a known protein that may be involved in a disease state or aberrant biological activity. Using the techniques for creating a fusion of gDS and proteins of interest such as Thy-1, JAM-2, E1, and gH outlined in EXAMPLES 1 and 7-9 above, the polypeptide fragment of the invention can be fused to additional polypeptides of interest to alter their cell surface expression.

The nucleotide sequence corresponding to the protein of interest is inserted into a vector, such as the pCR3 plasmid utilized previously, as well as other vectors including but not limited to pMAM, pDR2, pBK, pSV2neo, pSV2hygro, in addition to any vector that can be used for these purposes possessing a DNA promoter (e.g., CMV, MMLV-LTR), a selectivity marker (e.g. neomycin, G418) and a cloning insertion site. The gDL polypeptide fragment is inserted into the vector as outlined above with the protein of interest to create a protein/gDS fusion.

The resulting vector is transfected into a mammalian cell line including for example, but not limited to 293T, 293 EBNA, HEK293, NIH3T3, COS, HeLa, and several tumor derived cell lines to assess mode of cell surface expression or level of secretion. Expression vectors of this type are particularly useful in gene therapy.

Optionally, the construct encoding the gDS polypeptide fragment fusion is further modified by methods familiar to one skilled in the art to add a genetic tag for aid in protein purification, including, for example, a histidine tag, a FLAG tag, and other such methods as outlined in Current Protocols in Molecular Biology (16.11,16.22), the disclosure of which is incorporated by reference.

### EXAMPLE 11

Definition of the Amino Acid Sequence Required For Secretion

To further analyze the gDS segment and more precisely define the amino acid sequence responsible for the release of polypeptides into the medium, additional constructs were prepared in which different fragments of the gD tag are expressed as fusion protein with the Thy-1 antigen, these fragments including gDS1 (amino acids 234-253, SEQ. ID NO: 36); gDS2 (amino acids 254-294, SEQ. ID NO: 40); gDS2-N (similar to gDS2 but the site for N-glycosylation is destroyed by replacing the Asn at position 262 by a Gin, SEQ. ID NO: 44); gD2S2-N (amino acids 254-294 of gD of herpes simplex virus type 2 with replacement of the Asn at position 262 by a Gin, SEQ. ID NO: 56); and gDS-PD (amino acids 254-275 wherein the site for N-glycosylation is destroyed by replacing the Asn at position 262 by a Gln, SEQ. ID NO: 48).

The vector pCR-3/LP Thy-1/gDS was generated by inserting the Thy-1/gDS fragment into the HindIII and XhoI restriction sites of the pCR-3 vector described above. The oligonucleotide primers used in the PCR reaction are set out in SEQ. ID NO: 31 and SEQ. ID NO: 32.

The four constructs, pCR-3/LP Thy-1/gDS1, pCR-3/LP Thy-1/gDS2, pCR-3/LP Thy-1/gDS2-N and pCR-3/LP Thy-1/gDS-PD were all derived from pCR-3 in a two step procedure. First, the nucleotide sequence encoding the Thy-1 antigen (amino acids −19 to 114), including its leader peptide and 10 nucleotides of 5′ UTR and lacking the hydrophobic C-terminal encoding domain, was amplified using Thy-1/gDS as template with the primers set out in SEQ. ID NO 33 and SEQ. ID NO: 34. The resulting amplified fragment was inserted into the Hind III and EcoRV restriction sites of the pCR-3 vector.

In a second step, the four different gD fragments were amplified by PCR using Thy-1/gDS as a template and the following oligonucleotide pairs as primers: pCR-3/LP Thy-1/gDS1 (SEQ. ID NO: 37 and SEQ. ID NO: 38); pCR-3/LP Thy-1/gDS2 (SEQ. ID NO: 41 and SEQ. ID NO: 42); pCR-3/LP Thy-1/gDS2-N (SEQ. ID NO: 45 and SEQ. ID NO: 46); pCR-3/LP Thy-1/gDS-PD (SEQ. ID NO: 49 and SEQ. ID NO: 50). These four fragments were inserted into the vector using the EcoRV and XhoI restriction sites.

To test if gD of HSV2 (designated gD2) also contains the functional secretory region identified in HSV1 gD, the construct pCR-3/LP Thy-1/gD2S2-N was prepared following a similar strategy except that gD of the HSV2 fragment was amplified using the gD2 coding region as template and the oligonucleotides SEQ. ID NO: 45 and SEQ. ID NO: 46 as primers.

The expression construct pCR-3/LP Thy-1 was derived from pCR-3/LP Thy-1/gDS1 by deletion of the complete Thy-1 coding sequence using HindIII and EcoRV restriction digestion and replacement with a Thy-1 sequence containing a stop codon after amino acid 114. This fragment was obtained by PCR using Thy-1/gDS as template and the oligonucleotides SEQ. ID NO: 59 and SEQ. ID NO: 60 as primers.

These different constructs as well as two control plasmids expressing either Thy-1 alone without its C-terminal hydrophobic domain or Thy-1 fused to the full length gDS tag were transfected in HEK293T cells. The culture medium was collected 2 days after transfection was analyzed by Western blot using an anti-Thy-1 antibody.

Western blot analysis shows that all the truncated gDS constructs increased the level of secretion of Thy-1 when compared to the full length gDS tag. The results indicated a greater increase in secretion with the amino acid sequence 254-294 (gDS2) compared to the full length tag sequence (gDS) or to the sequence 234-253 (gDS 1). Deletion of the N-glycosylation site by gDS2-N also appeared to increase secretion of the fusion protein to levels comparable to gDS2 addition, whereas reduction of the gD tag sequence length to the 22 amino acid gDS-PD sequence (amino acids 254-275 lacking glycosylation site) resulted in a slight diminution of the level of protein secretion when compared to gDS2-N. The sequence from the HSV type 2 gD (gD2S2-N) acted exactly like that of HSV1 despite a few amino acid differences between the gD sequences. Although the Thy-1 antigen without its hydrophobic C-terminal domain was also secreted, secretion of this antigen was increased when associated to gDS2, to gDS2-N, gDS-PD or gD2S2, suggesting that the presence of a sequence containing gDS2 (amino acids 254-294) is sufficient to increase secretion.

### EXAMPLE 12

gDS Fused to the Rubella Virus C Protein

Similar experiments were performed using the complete gD tag sequence (gDS) or the gDS2-N and gDS-PD sequences fused to the rubella C protein. The rubella virus nucleocapsid C protein has been shown to require the presence of either E1 or E2 in order to be released from the Golgi complex (Baron et al., J Gen Virol. 73:1073-86. 1992). These experiments were carried out to determine if the secretory signals could circumvent this E1/E2 requirement.

The vectors pCR-3/LP C/ and PCR-3/LP C/gDS encoding the C protein were derived from the PCR-3/gDS in two steps. First, the Thy-1 leader peptide (amino acids −19 to 4) and 10 nucleotides of the 5′ UTR were amplified by PCR using Thy-1/gDS as template and the primers set out in SEQ. ID NO: 23 and SEQ. ID NO: 24 as primers. This amplified fragment was inserted into the HindIII and BamHI sites of the pCR-3/gDS. The two constructs pCR-3/LP C/gDS2-N and pCR-3/LP C/gDS-PD were derived from pCR-3/LP Thy-1/gDS2-N and pCR-3/LP Thy-1/gDS-PD. The complete Thy-1 sequence was removed by HindIII/EcoRV digestion and replacement by the HindIII/EcoRV fragment of PCR-3/LP C/gDS. The samples were analyzed by SDS-PAGE and immunoblotting. Approximately 2.5×10⁴ cells or 0.1 ml of TCA-precipitated medium were suspended in 10 ml of sample buffer and heated 5 minutes at 95° C. before loading onto 10% or 12% SDS polyacrylamide gels and subjected to electrophoresis. Immunoblotting was carried out according to previously described procedures (Beghdadi-Rais et al., 1993, supra). The antibodies used were rabbit antisera to mouse Thy-1 antigen (MacDonald et al., 1985) or the gD tag (HSV1 gD amino acids 234-294) (Eurogentec, Herstal, Belgium), or mouse antiserum to rubella virus C protein (Chemicon, International, Temecula, Calif.). The C protein without its C-terminal hydrophobic domain (amino acids 2-280) was amplified from a vector containing the complete rubella genome, pSP/E1/E2/C, and inserted into the BamHI and EcoRV sites of the vector described above and transfected into HEK293T cells. To obtain pCR-3/LP C, a stop codon was added at the end of the C sequence, which was not the case for pCR-3/LP C/gDS. The primers used in these reactions were SEQ. ID NO 63, SEQ. ID NO: 64 and SEQ. ID NO: 65.

Western blot analysis using anti-C protein antibodies demonstrated that the C protein without the gD tag was present only intracellularly and was poorly secreted, if at all. Addition of the full length gD tag (gDS) to this protein enabled its secretion in the culture medium. The results obtained with the two constructs C/gDS2-N and C/gDS-PD confirmed the results obtained with the Thy-1 constructs, i.e. better secretion with the gDS2-N tag than with the either gDS or gDS-PD addition. However, the presence of gDS-PD on the C protein was sufficient to obtain secretion of the C protein, although not to the level of secretion observed by addition of the gDS2-N fragment. Taken together, these results not only demonstrated that the gDS2-N segment was necessary to obtain high level secretion of a polypeptide such as the C protein, but also that the 22 amino acid segment of gDS-PD was sufficient to circumvent the intracellular retention of the C protein.

### EXAMPLE 13

Intracellular Cleavage of the gD Tag by Furin

Furin, a proteolytic enzyme of the serine protease family, functions in cleavage of a broad spectrum of precursor proteins (e.g. insulin proreceptor or HIV glycoprotein 160) in the Golgi compartment resulting in the release of functional, mature proteins (Bravo, et al. J. Biol. Chem. 41: 25830-37. 1994). In this experiment, it is demonstrated that a furin cleavage site inserted between the Thy-1 antigen and the gDS tag results in intracellular fusion protein cleavage by endogenous furin enzyme after the amino acid sequence RSKR, and release of the Thy-1 antigen lacking the secretory signal tag in the culture medium.

For this experiment, plasmid pCR-3/LP Thy-1/fur/gDS2-N, derived from pCR-3/LP Thy-1/gDS2-N, was generated by deletion of the Thy-1 sequence using HindIII and EcoRV restriction sites and replacement by a furin cleavage site (RSKR) followed by a “linker” of 5 glycine residues (SEQ. ID NOs.: 51 and 52). This fragment was obtained by PCR using Thy-1/gDS as template and the oligonucleotides set out in SEQ. ID NO: 53 and SEQ. ID NO: 54 as primers. The samples were analyzed by SDS-PAGE and immunoblotting as described in previous Examples. The two constructs, Thy-1/gDS2-N and Thy-1/fur/gDS2-N were transfected into HEK 293T cells and the cell lysates and supernatants collected 2 days post-transfection and analyzed by Western blot using mouse anti-Thy-1 and anti-gd tag antibodies (HSV1 gD amino acids 234-294) (Eurogentec).

The two fusion proteins, Thy-1/gDS2-N and Thy-1/fur/gDS2-N, were detected in the cell supernatant with both anti-Thy-1 and anti-gD antibodies both at molecular weights of approximately 35 kDa. An additional band corresponding to the Thy-1 antigen without the gD tag was detected only in the supernatant of cells transfected with the Thy-1/fur/gDS2-N construct and was only visible with the anti-Thy-1 antibody, consistent with the idea that no gD tag is present on this protein.

### EXAMPLE 14

Development of Gene Therapy Vector Based on gDS Sequence

The construct created in Example 10 above encoding a protein of interest is used as a potential means of treatment for numerous diseases resulting from the aberrant expression of a gene of interest, as has been practiced for disease models such as cystic fibrosis (Koehler et al, Mol Ther. 4:84-91. 2001, the disclosure of which is incorporated by reference), cancer (Heideman et al, Cancer Gene Ther. 8:342-51. 2001, the disclosure of which is incorporated by reference), and diabetes (Lee et al, Nature 408:483-8. 2000, the disclosure of which is incorporated by reference). The gDL-, gD10-, gDS-, or fragments of gDS such as gDS1-, gDS2-, gDS2-N- and gDS-PD-fusion constructs are employed to generate expression of a protein of interest in a vector that is used in gene therapy. Delivery of the gDS-fusion constructs to appropriate cells is effected ex vivo, in situ, or in vivo by the use of vectors, such vectors including but not limited to adenoviral vectors and retroviral vectors. Ex-vivo, physical DNA-gene transfer methods include liposome and cationic liposome technology (Banerjee et al, J Biomater Appl. 16:3-21. 2001; Kageshita et al, Melanoma Res. 11:337-342. 2001, the disclosures of which are incorporated by reference) and other chemical treatments. See, for example, Anderson, Nature, supplement to vol. 392, no. 6679, pp.25-20 (1998). For additional reviews of gene therapy technology see Friedmann, Science, 244: 1275-1281 (1989); Verma, Scientific American: 68-84 (1990); and Miller, Nature, 357: 455-460 (1992).

The invention thus provides gene therapy to restore normal activity of the polypeptides of the invention; or to treat disease states involving polypeptides of the invention. Introduction of any one of the nucleotides of the present invention or a gene encoding the polypeptides of the present invention can also be accomplished with extrachromosomal substrates (transient expression) or artificial chromosomes (stable expression). Cells may also be cultured ex vivo in the presence of proteins of the present invention in order to proliferate or to produce a desired effect on or activity in such cells. Treated cells can then be introduced in vivo for therapeutic purposes. Alternatively, it is contemplated that in other human disease states, preventing the expression of or inhibiting the activity of polypeptides of the invention will be useful in treating the disease states.

We claim:

1. A purified and isolated polynucleotide encoding a secretory signal polypeptide, wherein said polypeptide is a non-cell surface anchoring amino terminal fragment,of sequence HSV-1 gDL (SEQ ID NO: 6) lacking at least 40 carboxy terminal amino acid residues, and substitution variants thereof that retain secretory activity.

2. The polynucleotide of claim 1 consisting essentially of sequence HSV-1 gDS (SEQ ID NO: 29)

3. The polynucleotide of claim 1 consisting essentially of sequence HSV-1 gDS2-N (SEQ ID NO: 43)

4. A purified and isolated polynucleotide encoding a secretory polypeptide consisting essentially of sequence HSV-1 gDS1 (SEQ ID NO: 35) and substitution variants thereof that retain secretory activity.

5. A purified and isolated polynucleotide encoding a secretory polypeptide consisting essentially of sequence HSV-1 gDS2 (SEQ ID NO: 39) and substitution variants thereof that retain secretory activity.

6. A purified and isolated polynucleotide encoding a secretory polypeptide consisting essentially of sequence HSV-1 gDS-PD (SEQ ID NO: 47) and substitution variants thereof that retain secretory activity.

7. A purified and isolated polynucleotide encoding a secretory polypeptide consisting essentially of sequence HSV-2 gDS (SEQ ID NO: 68) and substitution variants thereof that retain secretory activity.

8. The polynucleotide of claims 7 consisting essentially of HSV-2 gD2S2-N (SEQ ID NO: 55).

9. A polynucleotide encoding a secretory polypeptide selected from the group consisting of:
a) the polynucleotide according to any one of claims 1 through 8 and
b) a polynucleotide consisting essentially of a polypeptide coding region that specifically hybridizes to the secretory polypeptide coding region in the polynucleotide of (a) under conditions that include a final wash in 0.1×SSC and 0.1% SDS at 65° C.

10. The polynucleotide of claim 9 which is selected from the group consisting of gDS2-N (SEQ ID NO: 43), gD2S2-N (SEQ ID NO: 55), and HSV2 gDS (SEQ ID NO: 68).

11. A chimeric polynucleotide comprising the polynucleotide of claim 9 and a polynucleotide encoding a heterologous polypeptide.

12. The chimeric polynucleotide of claim 11 wherein the polynucleotide encoding a heterologous polypeptide is positioned 5′ to the polynucleotide of claim 9.

13. The chimeric polynucleotide of claim 11 wherein the polynucleotide encoding a heterologous polypeptide is positioned 3′ to the polynucleotide of claim 9.

14. The chimeric polynucleotide of claim 11 further comprising operatively-linked one or more expression regulatory elements 5′ to the chimeric polynucleotide coding region and a stop codon 3′ to the polynucleotide.

15. A chimeric polynucleotide comprising the polynucleotide of claim 9 and a heterologous polypeptide coding region and further comprising a peptide cleavage site coding region which is positioned in-frame between the heterologous polypeptide coding region.

16. An expression vector comprising the polynucleotide of claim 11, 12, 13, 14 or 15.

17. A host cell transformed or transfected with the expression vector of claim 16.

18. A method for expression of a secreted polypeptide comprising the steps of growing the host cell of claim 17 under conditions that permit expression and secretion of the heterologous polypeptide.

19. The method according to claim 18 further comprising the step of cleaving the secretory polypeptide from the heterologous polypeptide at a peptide cleavage site.
